(19) United States
(12) Patent Application Publication
(10) Pub. No.: US 2025/0051294 A1
(43) Pub. Date: Feb. 13, 2025

(54) METHOD FOR PREPARING CYCLOHEXYL THIAZOLYL PHENYL CARBAMATE

(71) Applicant: Cyteir Therapeutics, Inc., Stoneham, MA (US)

(72) Inventors: Kuangchu DAI, Changzhou (CN); Sebastien Pierre Louis ALAZET, Montreal (CA); Maxence Benjamin BOS, Saint-Laurent (CA); Felix CHAGNON, LaSalle (CA); Jean-Marc LAPIERRE, Merrimack, NH (US); Vu Linh LY, Montreal (CA); Bernhard Josef PAUL, Arlington, MA (US); Guangwei RONG, Changzhou (CN)

(21) Appl. No.: 18/798,938

(22) Filed: Aug. 9, 2024

(30) Foreign Application Priority Data
Aug. 11, 2023 (WO) PCT/CN2023/112435

(51) Int. Cl. C07D 277/22; B01J 31/12; B01J 31/28

(52) U.S. Cl. CPC C07D 277/22; B01J 31/122; B01J 31/28

(57) ABSTRACT

The present application relates to methods of preparing Compound A:

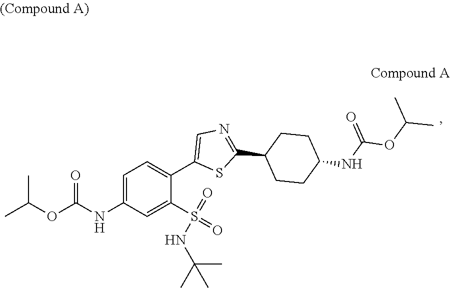 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt or solvate thereof.

### CROSS-REFERENCE TO RELATED APPLICATION

This application claims priority to, and the benefit of, International Patent Application No. PCT/CN2023/112435, filed on Aug. 11, 2023, the entire contents of which are incorporated herein by reference.

### BACKGROUND

Monocarboxylate transporters (MCTs) mediate influx and efflux of monocarboxylates, such as lactate, pyruvate, and ketone bodies, which play a central role in cellular metabolism and metabolic communications among tissues. Lactate is the end product of aerobic glycolysis, and has recently emerged as a critical regulator of cancer development, invasion, and metastasis. Tumor lactate levels correlate well with metastasis, tumor recurrence, and poor prognosis.

In order to avoid lactate-induced cytotoxicity, glycolytic cancer cells upregulate the expression of MCTs to increase their export capacity and avoid reaching toxic intracellular levels of lactate. Accordingly, compounds which modulate MCTs are useful for cancer treatment. Thus, there is a need for methods of preparing MCT modulator compounds with improved yield and purity, and desirable stereochemistry, at a reasonable cost. The present application addresses the need.

### SUMMARY

The present application relates to a method for preparing Compound A:

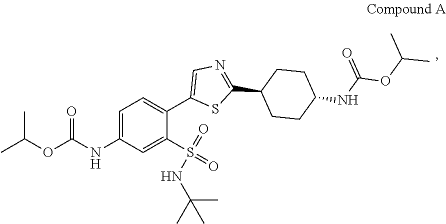 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt or solvate thereof, comprising Step (a) or Step (b):
Step (a): reacting Compound X with Compound Y to form Compound A:

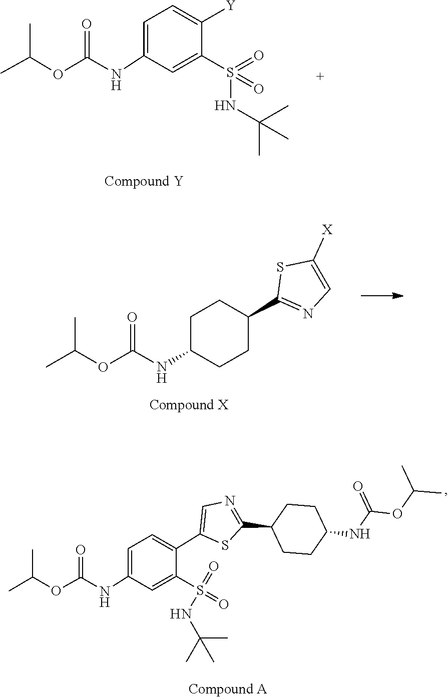

or
Step (b): reacting Compound Xₐₜ with Compound Yₐₜ to form Compound A:

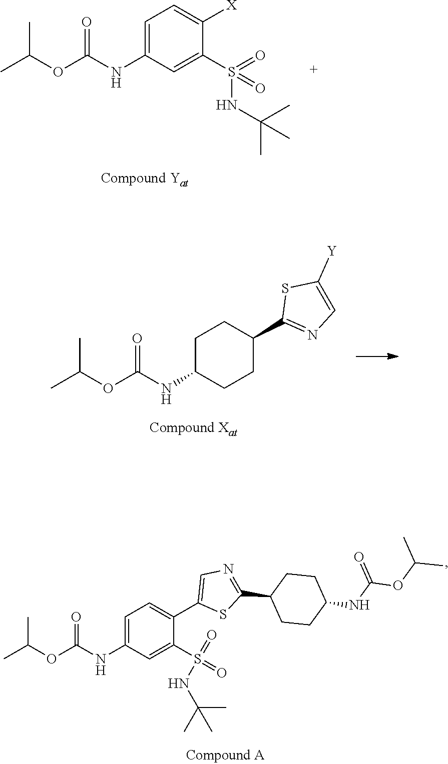

wherein X is —Cl, —Br, or —I and Y is

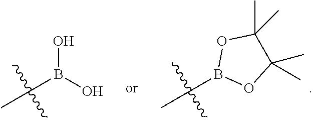

The present application relates to a method for preparing Compound A:

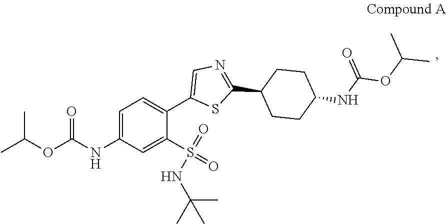 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt or solvate thereof, comprising Step A4:
Step A4: cyclization of Compound A4:

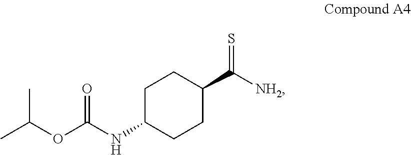 [SMILES: CC(C)OC(=O)N[C@H]1CC[C@H](C(N)=S)CC1]

to form Compound A5:

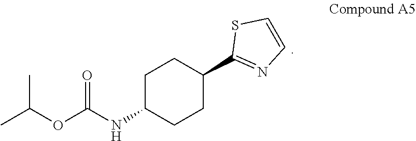 [SMILES: CC(C)OC(=O)N[C@H]1CC[C@H](c2nccs2)CC1]

The present application relates to a method for preparing Compound A:

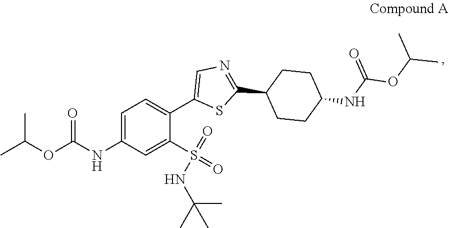 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt or solvate thereof, comprising Step A4:
Step A4: cyclization of Compound A4:

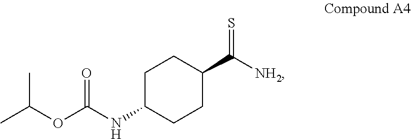 [SMILES: CC(C)OC(=O)N[C@H]1CC[C@H](C(N)=S)CC1]

to form Compound A5:

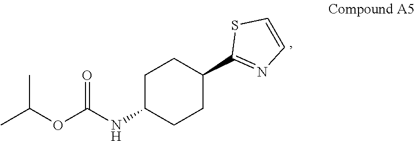 [SMILES: CC(C)OC(=O)N[C@H]1CC[C@H](c2nccs2)CC1]

wherein the cyclization of Compound A4 in the presence of an acid or a Lewis acid selected from ZnBr₂, Yb(OTf)₃·H₂O, Y(OTf)₃, Sc(OTf)₃, Zn(OTf)₂, FeCl₂, CoCl₂, p-toluene sulfonic acid, and camphor sulfonic acid, or a combination thereof. In some embodiments, the acid or Lewis acid is selected from ZnBr₂, Yb(OTf)₃·H₂O, Y(OTf)₃, and Sc(OTf)₃, or a combination thereof. In some embodiments, the acid or Lewis acid is ZnBr₂.

The present application relates to a method for preparing Compound A:

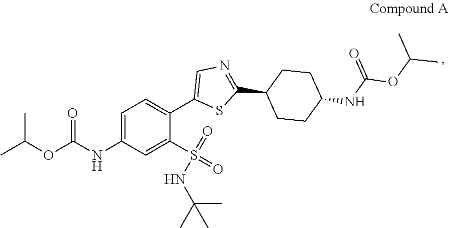 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt or solvate thereof, comprising Step A4:
Step A4: cyclization of Compound A4:

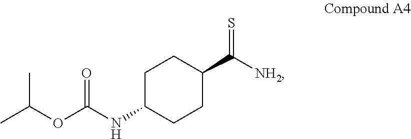 [SMILES: CC(C)OC(=O)N[C@H]1CC[C@H](C(N)=S)CC1]

to form Compound A5:

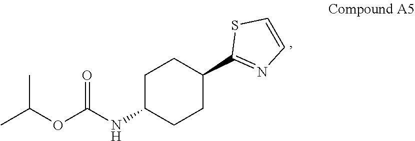 [SMILES: CC(C)OC(=O)N[C@H]1CC[C@H](c2nccs2)CC1]

wherein the cyclization of Compound A4 in the presence of 2-bromo-1,1-dimethoxy-ethane; 2-bromo-1,1-diethoxyethane; 2-bromoacetaldehyde; or 2-chloroacetaldehyde; or a combination thereof.

The present application relates to a method for preparing Compound A:

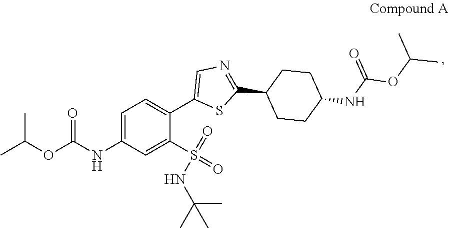 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt or solvate thereof, comprising Step A4, as follows:

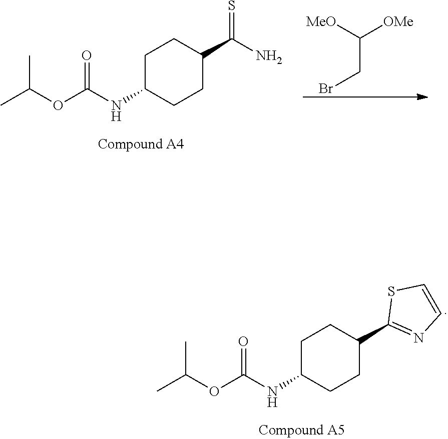

The present application also provides a pharmaceutical composition comprising Compound A or a pharmaceutically acceptable salt or solvate thereof prepared by the methods of the present application, and a pharmaceutically acceptable carrier or excipient.

The present application further provides a method of treating a disease or disorder, for example, cancer, an autoimmune disease, an immune deficiency, or a neurodegenerative disease. The method comprises administering to a subject in need thereof an effective amount of Compound A or a pharmaceutically acceptable salt or solvate thereof prepared by the methods of the present application or a pharmaceutical composition disclosed herein.

The present application provides use of Compound A or a pharmaceutically acceptable salt or solvate thereof that functions as modulator of MCT activity.

The present application provides a method of treating or preventing a disease or disorder associated with the abnormal expression or activity of monocarboxylate transporters, or dependency on the expression or activity of at least one MCT.

The present application further provides use of Compound A or a pharmaceutically acceptable salt or solvate thereof prepared by the methods of the present application, or a pharmaceutical composition disclosed herein, in the manufacture of a medicament for the treatment of a disease or disorder, for example, a cancer, an autoimmune disease, an immune deficiency, or a neurodegenerative disease.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art to the present application. In the case of conflict, the present specification, including definitions, will control. In addition, the materials, methods, and examples are illustrative only and are not intended to be limiting.

Other features and advantages of the disclosure will be apparent from the following detailed description and claims.

### DETAILED DESCRIPTION

### Methods of the Present Application

The present application provides a method of preparing Compound A:

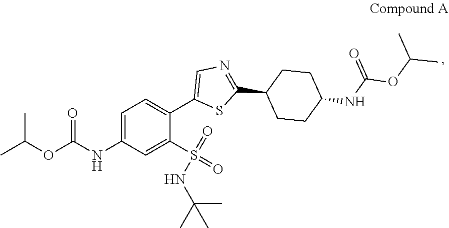 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt or solvate thereof.

Embodiment 1: The present application provides a method of preparing Compound A:

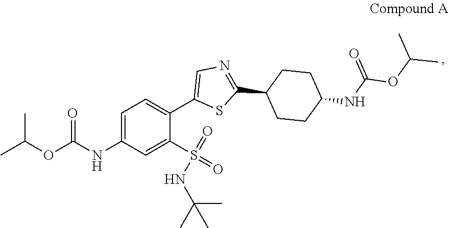 [SMILES: CC(C)OC(=O)Nc1ccc(-c2cnc([C@H]3CC[C@H](NC(=O)OC(C)C)CC3)s2)c(S(=O)(=O)NC(C)(C)C)c1]

or a pharmaceutically acceptable salt or solvate thereof, comprising Step (a) or Step (b):
Step (a): reacting Compound X with Compound Y to form Compound A:

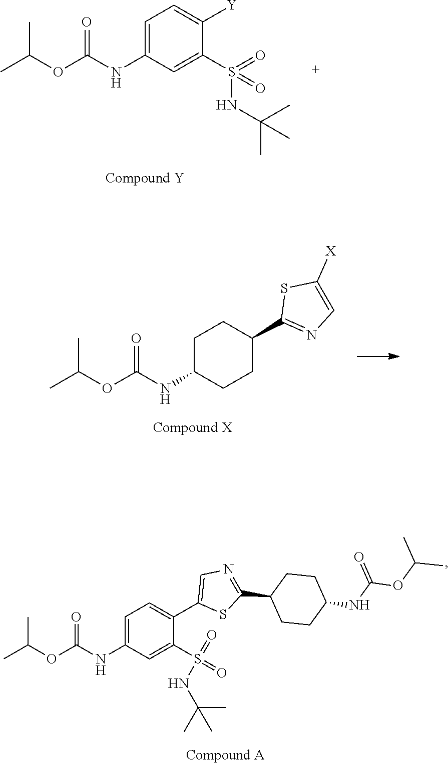

or
Step (b): reacting Compound Xₐₜ with Compound Yₐₜ to form Compound A:

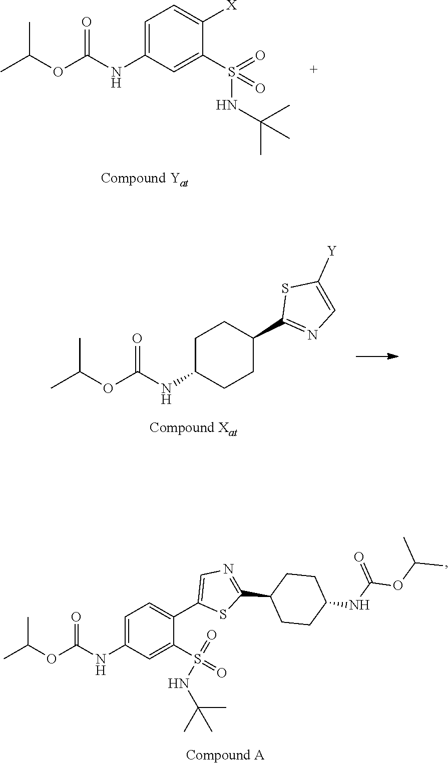

wherein X is —Cl, —Br, or —I and Y is

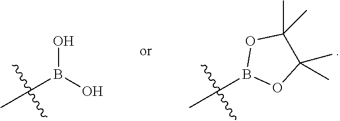

### Step (a)

In some embodiments, Step (a) comprises adding Compound Y to Compound X.

In some embodiments, Step (a) comprises adding a catalyst to Compound Y and Compound X. In some embodiments, Step (a) comprises adding a palladium catalyst to Compound Y and Compound X. In some embodiments, Step (a) comprises adding Bis(triphenylphosphine)palladium (II) dichloride (PdCl₂(PPh₃)₂) to Compound Y and Compound X. In some embodiments, Step (a) comprises adding a solvent to Compound Y and Compound X. In some embodiments, Step (a) comprises adding a polar aprotic solvent (e.g., dimethylformamide (DMF)) to Compound Y and Compound X. In some embodiments, Step (a) comprises adding DMF to Compound Y and Compound X. In some embodiments, Step (a) comprises adding DMF to PdCl₂(PPh₃)₂, Compound X, and Compound Y. In some embodiments, Step (a) comprises adding ethylene glycol to Compound X and Compound Y. In some embodiments, Step (a) comprises adding DMF and ethylene glycol to Compound X and Compound Y. In some embodiments, Step (a) comprises adding DMF and ethylene glycol to PdCl₂(PPh₃)₂, Compound X, and Compound Y. In some embodiments, Step (a) comprises adding DMF and ethylene glycol to PdCl₂(PPh₃)₂, Compound X, and Compound Y under nitrogen. In some embodiments, Step (a) comprises reaction in a solution of sodium carbonate. In some embodiments, Step (a) comprises adding a solution of sodium carbonate in water to the reaction mixture. In some embodiments, the solution of sodium carbonate is added slowly. In some embodiments, the solution of sodium carbonate is slowly added at a temperature between about 15° C. and about 35° C. In some embodiments, the solution of sodium carbonate is added at room temperature during about 1 hour. In some embodiments, the solution of sodium carbonate is added at a temperature between about 15° C. and about 35° C. during about 1 hour.

In some embodiments, Step (a) comprises stirring the reaction mixture at high temperature. In some embodiments, Step (a) comprises stirring the reaction mixture at a temperature below about 100° C. In some embodiments, Step (a) comprises stirring the reaction mixture at a temperature of about 50° C., about 55° C., about 60° C., about 65° C., about 70° C., about 75° C., about 80° C., about 85° C., about 90° C., about 95° C., or about 100° C. In some embodiments, Step (a) comprises stirring the reaction mixture at a temperature below about 100° C. under nitrogen atmosphere for about 6 hours, about 5 hours, about 4 hours, about 3 hours, about 2 hours, or about 1 hour.

In some embodiments, PdCl₂(PPh₃)₂, Compound X, and Compound Y are reacted at a temperature of about 50° C., about 55° C., about 60° C., about 65° C., about 70° C., about 75° C., about 80° C., about 85° C., about 90° C., about 95° C., or about 100° C. In some embodiments, PdCl₂(PPh₃)₂, Compound X, and Compound Y are reacted at a temperature of about 50° C., about 55° C., about 60° C., about 65° C., about 70° C., about 75° C., about 80° C., about 85° C., about 90° C., about 95° C., or about 100° C. for about 2 to 3 hours. In some embodiments, PdCl₂(PPh₃)₂, Compound X, and Compound Y are reacted at a temperature of about 50° C., about 55° C., about 60° C., about 65° C., about 70° C., about 75° C., about 80° C., about 85° C., about 90° C., about 95° C., or about 100° C. for about 2 to 3 hours under nitrogen atmosphere.

In some embodiments, Step (a) comprises adding water to the reaction mixture. In some embodiments, Step (a) comprises adding water to the reaction mixture at about room temperature. In some embodiments, Step (a) comprises adding water to the reaction mixture dropwise, at about room temperature. In some embodiments, Step (a) comprises adding water to the reaction mixture during about 3 hours, at about room temperature.

In some embodiments after addition of water, Step (a) comprises stirring the reaction mixture for less than about 36 hours. In some embodiments after addition of water, Step (a) comprises stirring the reaction mixture for less than about 30 hours, less than about 25 hours, less than about 20 hours, or less than about 15 hours. In some embodiments after addition of water, Step (a) comprises stirring the reaction mixture for about 20 hours.

In some embodiments, Step (a) comprises filtering the resultant solid, Compound A. In some embodiments, Step (a) comprises filtering and collecting the resultant solid (i.e., Compound A). In some embodiments, the filter cake is washed. In some embodiments, the filter cake is washed with water. In some embodiments, the filter cake is washed with methyl tert-butyl ether. In some embodiments, the filter cake is washed with water and methyl tert-butyl ether. In some embodiments, the washed solid (i.e., Compound A) is dried under vacuum.

In some embodiments, Step (a) comprises adding 3-mercaptopropyl ethyl sulphide silica, low cross linking (SPM32) resin to Compound A. In some embodiments, Step (a) comprises suspending SPM32 resin and Compound A in 2-MeTHF (2-Methyltetrahydrofuran). In some embodiments, Step (a) comprises suspending SPM32 resin and Compound A in 2-MeTHF at high temperature. In some embodiments, Step (a) comprises suspending SPM32 resin and Compound A in 2-MeTHF at a temperature below about 100° C. In some embodiments, Step (a) Step (a) comprises suspending SPM32 resin and Compound A in 2-MeTHF at a temperature of about 50° C., about 55° C., about 60° C., about 65° C., about 70° C., about 75° C., about 80° C., about 85° C., about 90° C., about 95° C., or about 100° C. In some embodiments, Step (a) comprises suspending SPM32 resin and Compound A in 2-MeTHF at a temperature below about 100° C. and stirring the suspension for about 24 hours.

In some embodiments, the solution of Compound A and resin is filtered. In some embodiments, the filter cake is rinsed to provide a 2-MeTHF solution of Compound A. In some embodiments, the filter cake is rinsed with water to provide a 2-MeTHF solution of Compound A. In some embodiments, the filter cake is rinsed with water to provide a 2-MeTHF solution of Compound A.

In some embodiments, the 2-MeTHF solution of Compound A is concentrated. In some embodiments, the 2-MeTHF solution of Compound A is concentrated to about 4-6 volumes to form a concentrated solution of 2-MeTHF and Compound A. In some embodiments, acetonitrile is added to the concentrated solution of 2-MeTHF and Compound A. In some embodiments, the resultant acetonitrile solution is concentrated to about 9-11 volumes to form a slurry. In some embodiments, the resultant acetonitrile solution is concentrated to about 9-11 volumes and another portion of acetonitrile is added and the resultant acetonitrile solution is concentrated to about 9-11 volumes to form a slurry.

In some embodiments, the slurry is heated to a temperature below about 100° C. In some embodiments, the slurry solution is heated at a temperature of about 50° C., about 55° C., about 60° C., about 65° C., about 70° C., about 75° C., about 80° C., about 85° C., about 90° C., about 95° C., or about 100° C. In some embodiments, the slurry is heated to a temperature below about 100° C. to form a dissolved solution of Compound A. In some embodiments, the slurry solution is heated at a temperature of about 50° C., about 55° C., about 60° C., about 65° C., about 70° C., about 75° C., about 80° C., about 85° C., about 90° C., about 95° C., or about 100° C. to form a dissolved solution of Compound A.

In some embodiments, the dissolved solution of Compound A is cooled to a temperature of about 45-55° C. In some embodiments, pure Compound A is added to the dissolved solution of Compound A. In some embodiments, pure Compound A is added to dissolved solution of Compound A at a temperature of about 45-55° C. In some embodiments, the resultant precipitate is filtered. In some embodiments, the resultant precipitate is filtered and collected. In some embodiments, the resultant precipitate is filtered, the filter cake washed with acetonitrile (MeCN) and Compound A is collected. In some embodiments, Step (a) may further comprises drying the solid, Compound A (e.g., under vacuum).

In some embodiments, the yield of Compound A is at least about 60%, at least about 65%, at least about 70%, at least about 75%, at least about 80%, at least about 85%, or at least about 90%. In some embodiments, the yield of Compound A is at least about 75%.

In some embodiments, the purity of Compound A is at least about 70%, at least about 75%, at least about 80%, at least about 85%, at least about 85%, at least about 90%, at least about 95%, at least about 98%, or at least about 99%. In some embodiments, the purity of Compound A is at least about 99%.

### Step (b)

In some embodiments, Step (b) comprises adding Compound Yₐₜ to Compound X at to form Compound A.

In some embodiments, Step (b) comprises adding a catalyst to Compound Yₐₜ and Compound Xₐₜ to form Compound A. In some embodiments, Step (b) comprises adding a palladium catalyst to Compound Yₐₜ and Compound Xₐₜ to form Compound A. In some embodiments, Step (b) comprises adding a solvent to Compound Yₐₜ and Compound Xₐₜ to form Compound A. In some embodiments, Step (b) comprises adding a base to the catalyst, Compound Yₐₜ and Compound Xₐₜ to form Compound A. In some embodiments, Step (b) comprises adding a base to the palladium catalyst, Compound Yₐₜ and Compound Xₐₜ to form Compound A.

In some embodiments, Step (b) comprises adding a phosphine ligand to the base, the catalyst, Compound Yₐₜ and Compound Xₐₜ to form Compound A. In some embodiments, Step (b) comprises adding a phosphine ligand to the base, the palladium catalyst, Compound Yₐₜ and Compound Xₐₜ to form Compound A.

In some embodiments, Embodiment 1 comprises the use of boric acid as the catalyst for the coupling reaction.

Embodiment 2: In some aspects, the present application provides a method of preparing Compound A comprising Step (a).

Embodiment 3: In some aspects, the present application provides a method of preparing Compound A comprising Step (a), wherein Step (a) is Step (a1):

Step (a1): reacting Compound X with Compound B6 to form Compound A:

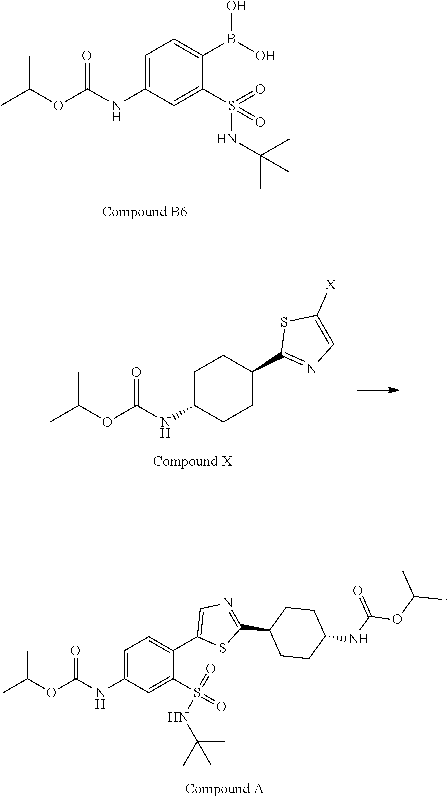

### Step (a1)

In some embodiments, Step (a1) comprises adding Compound B6 to Compound X.

In some embodiments, Step (a1) comprises adding a catalyst to Compound B6 and Compound X. In some embodiments, Step (a1) comprises adding Bis(triphenylphosphine)palladium (II) dichloride (PdCl₂(PPh₃)₂) to Compound B6 and Compound X. In some embodiments, Step (a1) comprises adding a solvent to Compound B6 and Compound X. In some embodiments, Step (a1) comprises adding a polar aprotic solvent (e.g., DMF) to Compound B6 and Compound X. In some embodiments, Step (a1) comprises adding DMF to Compound B6 and Compound X. In some embodiments, Step (a1) comprises adding DMF to PdCl₂(PPh₃)₂, Compound X, and Compound B6. In some embodiments, Step (a1) comprises adding ethylene glycol to Compound X and Compound B6. In some embodiments, Step (a1) comprises adding DMF and ethylene glycol to Compound X and Compound B6. In some embodiments, Step (a1) comprises adding DMF and ethylene glycol to PdCl₂(PPh₃)₂, Compound X, and Compound B6. In some embodiments, Step (a1) comprises adding DMF and ethylene glycol to PdCl₂(PPh₃)₂, Compound X, and Compound B6 under nitrogen. In some embodiments, Step (a1) comprises reaction in a solution of sodium carbonate. In some embodiments, Step (a1) comprises adding a solution of sodium carbonate in water to the reaction mixture. In some embodiments, the solution of sodium carbonate is added slowly. In some embodiments, the solution of sodium carbonate is slowly added at a temperature between about 15° C. and about 35° C. In some embodiments, the solution of sodium carbonate is added at room temperature during about 1 hour. In some embodiments, the solution of sodium carbonate is added at a temperature between about 15° C. and about 35° C. during about 1 hour.

In some embodiments, Step (a1) comprises stirring the reaction mixture at high temperature. In some embodiments, Step (a1) comprises stirring the reaction mixture at a temperature below about 100° C. In some embodiments, Step (a1) comprises stirring the reaction mixture at a temperature of about 50° C., about 55° C., about 60° C., about 65° C., about 70° C., about 75° C., about 80° C., about 85° C., about 90° C., about 95° C., or about 100° C. In some embodiments, Step (a1) comprises stirring the reaction mixture at a temperature below about 100° C. under nitrogen atmosphere for about 6 hours, about 5 hours, about 4 hours, about 3 hours, about 2 hours, or about 1 hour.

In some embodiments, PdCl₂(PPh₃)₂, Compound X, and Compound B6 are reacted at a temperature of about 50° C., about 55° C., about 60° C., about 65° C., about 70° C., about 75° C., about 80° C., about 85° C., about 90° C., about 95° C., or about 100° C. In some embodiments, PdCl₂(PPh₃)₂, Compound X, and Compound B6 are reacted at a temperature of about 50° C., about 55° C., about 60° C., about 65° C., about 70° C., about 75° C., about 80° C., about 85° C., about 90° C., about 95° C., or about 100° C. for about 2 to 3 hours. In some embodiments, PdCl₂(PPh₃)₂, Compound X, and Compound B6 are reacted at a temperature of about 50° C., about 55° C., about 60° C., about 65° C., about 70° C., about 75° C., about 80° C., about 85° C., about 90° C., about 95° C., or about 100° C. for about 2 to 3 hours under nitrogen atmosphere.

In some embodiments, Step (a1) comprises adding water to the reaction mixture. In some embodiments, Step (a1) comprises adding water to the reaction mixture at about room temperature. In some embodiments, Step (a1) comprises adding water to the reaction mixture dropwise, at about room temperature. In some embodiments, Step (a1) comprises adding water to the reaction mixture during about 3 hours, at about room temperature.

In some embodiments after addition of water, Step (a1) comprises stirring the reaction mixture for less than about 36 hours. In some embodiments after addition of water, Step (a1) comprises stirring the reaction mixture for less than about 30 hours, less than about 25 hours, less than about 20 hours, or less than about 15 hours. In some embodiments after addition of water, Step (a1) comprises stirring the reaction mixture for about 20 hours.

In some embodiments, Step (a1) comprises filtering the resultant solid, Compound A. In some embodiments, Step (a1) comprises filtering and collecting the resultant solid (i.e., Compound A). In some embodiments, the filter cake is washed. In some embodiments, the filter cake is washed with water. In some embodiments, the filter cake is washed with methyl tert-butyl ether. In some embodiments, the filter cake is washed with water and methyl tert-butyl ether. In some embodiments, the washed solid (i.e., Compound A) is dried under vacuum.

In some embodiments, Step (a1) comprises adding SPM32 resin to Compound A. In some embodiments, Step (a1) comprises suspending SPM32 resin and Compound A in 2-MeTHF. In some embodiments, Step (a1) comprises suspending SPM32 resin and Compound A in 2-MeTHF at high temperature. In some embodiments, Step (a1) comprises suspending SPM32 resin and Compound A in 2-MeTHF at a temperature below about 100° C. In some embodiments, Step (a1) Step (a1) comprises suspending SPM32 resin and Compound A in 2-MeTHF at a temperature of about 50° C., about 55° C., about 60° C., about 65° C., about 70° C., about 75° C., about 80° C., about 85° C., about 90° C., about 95° C., or about 100° C. In some embodiments, Step (a1) comprises suspending SPM32 resin and Compound A in 2-MeTHF at a temperature below about 100° C. and stirring the suspension for about 24 hours.

In some embodiments, the solution of Compound A and resin is filtered. In some embodiments, the filter cake is rinsed to provide a 2-MeTHF solution of Compound A. In some embodiments, the filter cake is rinsed with water to provide a 2-MeTHF solution of Compound A. In some embodiments, the filter cake is rinsed with water to provide a 2-MeTHF solution of Compound A.

In some embodiments, the 2-MeTHF solution of Compound A is concentrated. In some embodiments, the 2-MeTHF solution of Compound A is concentrated to about 4-6 volumes to form a concentrated solution of 2-MeTHF and Compound A. In some embodiments, acetonitrile is added to the concentrated solution of 2-MeTHF and Compound A. In some embodiments, the resultant acetonitrile solution is concentrated to about 9-11 volumes to form a slurry. In some embodiments, the resultant acetonitrile solution is concentrated to about 9-11 volumes and another portion of acetonitrile is added and the resultant acetonitrile solution is concentrated to about 9-11 volumes to form a slurry.

In some embodiments, the slurry is heated to a temperature below about 100° C. In some embodiments, the slurry solution is heated at a temperature of about 50° C., about 55° C., about 60° C., about 65° C., about 70° C., about 75° C., about 80° C., about 85° C., about 90° C., about 95° C., or about 100° C. In some embodiments, the slurry is heated to a temperature below about 100° C. to form a dissolved solution of Compound A. In some embodiments, the slurry solution is heated at a temperature of about 50° C., about 55° C., about 60° C., about 65° C., about 70° C., about 75° C., about 80° C., about 85° C., about 90° C., about 95° C., or about 100° C. to form a dissolved solution of Compound A.

In some embodiments, the dissolved solution of Compound A is cooled to a temperature of about 45-55° C. In some embodiments, pure Compound A is added to the dissolved solution of Compound A. In some embodiments, pure Compound A is added to dissolved solution of Compound A at a temperature of about 45-55° C. In some embodiments, the resultant precipitate is filtered. In some embodiments, the resultant precipitate is filtered and collected. In some embodiments, the resultant precipitate is filtered, the filter cake washed with MeCN and Compound A is collected. In some embodiments, Step (a1) may further comprises drying the solid, Compound A (e.g., under vacuum).

In some embodiments, the yield of Compound A is at least about 60%, at least about 65%, at least about 70%, at least about 75%, at least about 80%, at least about 85%, or at least about 90%. In some embodiments, the yield of Compound A is at least about 75%.

In some embodiments, the yield of Compound A is at least about 60%, at least about 65%, at least about 70%, at least about 75%, at least about 80%, at least about 85%, or at least about 90%. In some embodiments, the yield of Compound A is at least about 75%.

In some embodiments, the purity of Compound A is at least about 70%, at least about 75%, at least about 80%, at least about 85%, at least about 85%, at least about 90%, at least about 95%, at least about 98%, or at least about 99%. In some embodiments, the purity of Compound A is at least about 99%.

Embodiment 4: In some aspects, in Embodiment 1 or 2, Step (a) is Step (a2):
Step (a2): reacting Compound X with Compound B6′ to form Compound A:

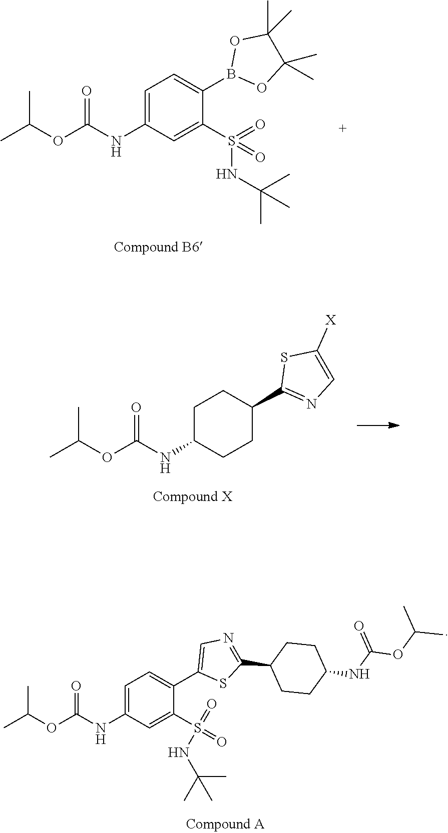

### Step (a2)

In some embodiments, Step (a2) comprises adding Compound B6′ to Compound X to form Compound A.

In some embodiments, Step (a2) comprises adding a catalyst to Compound B6′ and Compound X to form Compound A. In some embodiments, Step (a2) comprises adding a palladium catalyst to Compound B6′ and Compound X to form Compound A. In some embodiments, Step (a2) comprises adding a solvent to Compound B6′ and Compound X to form Compound A. In some embodiments, Step (a2) comprises adding a base to the catalyst, Compound B6′ and Compound X to form Compound A. In some embodiments, Step (a2) comprises adding a base to the palladium catalyst, Compound B6′ and Compound X to form Compound A.

In some embodiments, Step (a2) comprises adding a phosphine ligand to the base, the catalyst, Compound B6′ and Compound X to form Compound A. In some embodiments, Step (a2) comprises adding a phosphine ligand to the base, the palladium catalyst, Compound B6′ and Compound X to form Compound A.

In some embodiments, the yield of Compound A is at least about 60%, at least about 65%, at least about 70%, at least about 75%, at least about 80%, at least about 85%, or at least about 90%.

In some embodiments, the purity of Compound A is at least about 70%, at least about 75%, at least about 80%, at least about 85%, at least about 85%, at least about 90%, at least about 95%, at least about 98%, or at least about 99%.

Embodiment 5: In some aspects, in any one of Embodiments 1-4, X is —Br.

Embodiment 6: In some aspects, the method of any one of Embodiments 1-5 further comprises Step A4:

### Step A4: Cyclization of Compound A4:

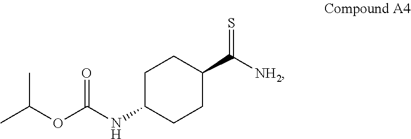 [SMILES: CC(C)OC(=O)N[C@H]1CC[C@H](C(N)=S)CC1]

to form Compound A5:

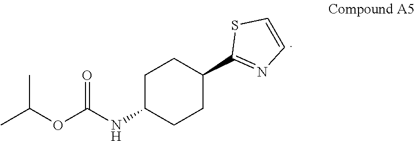 [SMILES: CC(C)OC(=O)N[C@H]1CC[C@H](c2nccs2)CC1]

### Step A4

In some embodiments, the present application provides the method of making Compound A5 or a pharmaceutically acceptable salt or solvate thereof. In some embodiments, the present application provides the method of making Compound A5.

In some embodiments, Compound A5 or a pharmaceutically acceptable salt or solvate thereof is prepared by a method disclosed herein. In some embodiments, Compound A5 is prepared by a method disclosed herein.

In some embodiments, Compound A5 is prepared by adding a 2-carbon unit cyclization reagent (e.g., 2-bromo-1,1-dimethoxyethane; 2-bromo-1,1-diethoxyethane; 2-bromoacetaldehyde; and 2-chloroacetaldehyde) to Compound A4.

In some embodiments, the method of the present application comprises:

Step A4: reacting Compound A4 with a 2-carbon unit cyclization reagent (e.g., 2-bromo-1,1-dimethoxyethane; 2-bromo-1,1-diethoxyethane; 2-bromoacetaldehyde; and 2-chloroacetaldehyde) to form Compound A5.

In some embodiments, Step A4 comprises adding a 2-carbon unit cyclization reagent (e.g., 2-bromo-1,1-dimethoxyethane) to Compound A4.

In some embodiments, Step A4 comprises adding an acid or a Lewis acid to Compound A4. In some embodiments, Step A4 comprises adding zinc bromide (ZnBr₂) to Compound A4. In some embodiments, Step A4 comprises adding a solvent to Compound A4. In some embodiments, Step A4 comprises adding a polar protic solvent (e.g., acetonitrile and water) to Compound A4. In some embodiments, Step A4 comprises adding acetonitrile and water to Compound A4.

In some embodiments, Step A4 comprises adding 2-bromo-1,1-dimethoxyethane to Compound A4 in acetonitrile and water. In some embodiments, Step A4 comprises adding 2-bromo-1,1-dimethoxyethane followed by an acid or a Lewis acid to Compound A4 in acetonitrile and water. In some embodiments, Step A4 comprises reacting Compound A4 with 2-bromo-1,1-dimethoxyethane in the presence of an acid or a Lewis acid at a temperature below about 100° C. In some embodiments, Compound A4 is reacted with 2-bromo-1,1-dimethoxyethane in the presence of an acid or a Lewis acid at a temperature of about 80° C. In some embodiments, Compound A4 is reacted with 2-bromo-1,1-dimethoxyethane in the presence of an acid or a Lewis acid at a temperature of about 50° C., about 55° C., about 60° C., about 65° C., about 70° C., about 75° C., about 80° C., about 85° C., about 90° C., about 95° C., or about 100° C.

In some embodiments, Step A4 comprises adding 2-bromo-1,1-dimethoxyethane followed by zinc bromide to Compound A4 in acetonitrile and water. In some embodiments, Step A4 comprises reacting Compound A4 with 2-bromo-1,1-dimethoxyethane in the presence of zinc bromide at a temperature below about 100° C. In some embodiments, Compound A4 is reacted with 2-bromo-1,1-dimethoxyethane in the presence of zinc bromide at a temperature of about 80° C. In some embodiments, Compound A4 is reacted with 2-bromo-1,1-dimethoxyethane in the presence of zinc bromide at a temperature of about 50° C., about 55° C., about 60° C., about 65° C., about 70° C., about 75° C., about 80° C., about 85° C., about 90° C., about 95° C., or about 100° C.

In some embodiments, Step A4 comprises reacting Compound A4 with 2-bromo-1,1-dimethoxyethane in the presence of an acid or a Lewis acid for less than about 24 hours. In some embodiments, Compound A4 is reacted with 2-bromo-1,1-dimethoxyethane in the presence of an acid or a Lewis acid for less than about 20 hours, less than about 15 hours, less than about 10 hours, or less than about 9 hours. In some embodiments, Compound A4 is reacted with 2-bromo-1,1-dimethoxyethane in the presence of an acid or a Lewis acid for about 12 hours, about 11 hours, about 10 hours, about 9 hours, about 8 hours, about 7 hours, about 6 hours, about 5 hours, about 4 hours, or about 3 hours. In some embodiments, Compound A4 is reacted with 2-bromo-1,1-dimethoxyethane in the presence of an acid or a Lewis acid for about 5-9 hours.

In some embodiments, Step A4 comprises reacting Compound A4 with 2-bromo-1,1-dimethoxyethane in the presence of an acid or a Lewis acid (e.g., zinc bromide, ytterbium triflate, iron chloride, scandium triflate, copper triflate, zinc triflate, barium triflate, samarium triflate, indium triflate, cerium triflate, tin triflate, iron triflate, calcium triflate, aluminum triflate, scandium triflate, yttrium triflate, zinc acetate, zinc chloride, cobalt chloride, zinc iodide, cerium chloride, ytterbium chloride, titanium tetrachloride, boron trifluoride etherate, p-toluene sulfonic acid (p-TSA), pyridium p-toluene sulfonate (PPTS), acetic acid, trifluoroacetic acid (TFA), methane sulfonic acid (MSA), trifluoromethanesulfonic acid (TFA), camphor sulfonic acid (CSA), hydrochloric acid, and hydrobromic acid) for less than about 24 hours. In some embodiments, Compound A4 is reacted with 2-bromo-1,1-dimethoxyethane in the presence of an acid or a Lewis acid (e.g., zinc bromide, ytterbium triflate, iron chloride, scandium triflate, copper triflate, zinc triflate, barium triflate, samarium triflate, indium triflate, cerium triflate, tin triflate, iron triflate, calcium triflate, aluminum triflate, scandium triflate, yttrium triflate, zinc acetate, zinc chloride, cobalt chloride, zinc iodide, cerium chloride, ytterbium chloride, titanium tetrachloride, boron trifluoride etherate, p-toluene sulfonic acid (p-TSA), pyridium p-toluene sulfonate (PPTS), acetic acid, trifluoroacetic acid (TFA), methane sulfonic acid (MSA), trifluoromethanesulfonic acid (TFA), camphor sulfonic acid (CSA), hydrochloric acid, and hydrobromic acid) for less than about 20 hours, less than about 15 hours, less than about 10 hours, or less than about 9 hours. In some embodiments, Compound A4 is reacted with 2-bromo-1,1-dimethoxyethane in the presence of an acid or a Lewis acid (e.g., zinc bromide, ytterbium triflate, iron chloride, scandium triflate, copper triflate, zinc triflate, barium triflate, samarium triflate, indium triflate, cerium triflate, tin triflate, iron triflate, calcium triflate, aluminum triflate, scandium triflate, yttrium triflate, zinc acetate, zinc chloride, cobalt chloride, zinc iodide, cerium chloride, ytterbium chloride, titanium tetrachloride, boron trifluoride etherate, p-toluene sulfonic acid (p-TSA), pyridium p-toluene sulfonate (PPTS), acetic acid, trifluoroacetic acid (TFA), methane sulfonic acid (MSA), trifluoromethanesulfonic acid (TFA), camphor sulfonic acid (CSA), hydrochloric acid, and hydrobromic acid) for about 12 hours, about 11 hours, about 10 hours, about 9 hours, about 8 hours, about 7 hours, about 6 hours, about 5 hours, about 4 hours, or about 3 hours. In some embodiments, Compound A4 is reacted with 2-bromo-1,1-dimethoxyethane in the presence of an acid or a Lewis acid (e.g., zinc bromide, ytterbium triflate, iron chloride, scandium triflate, copper triflate, zinc triflate, barium triflate, samarium triflate, indium triflate, cerium triflate, tin triflate, iron triflate, calcium triflate, aluminum triflate, scandium triflate, yttrium triflate, zinc acetate, zinc chloride, cobalt chloride, zinc iodide, cerium chloride, ytterbium chloride, titanium tetrachloride, boron trifluoride etherate, p-toluene sulfonic acid (p-TSA), pyridium p-toluene sulfonate (PPTS), acetic acid, trifluoroacetic acid (TFA), methane sulfonic acid (MSA), trifluoromethanesulfonic acid (TFA), camphor sulfonic acid (CSA), hydrochloric acid, and hydrobromic acid) for about 5-9 hours.

In some embodiments, Step A4 comprises reacting Compound A4 with 2-bromo-1,1-dimethoxyethane in the presence of zinc bromide for less than about 24 hours. In some embodiments, Compound A4 is reacted with 2-bromo-1,1-dimethoxyethane in the presence of zinc bromide for less than about 20 hours, less than about 15 hours, less than about 10 hours, or less than about 9 hours. In some embodiments, Compound A4 is reacted with 2-bromo-1,1-dimethoxyethane in the presence of zinc bromide for about 12 hours, about 11 hours, about 10 hours, about 9 hours, about 8 hours, about 7 hours, about 6 hours, about 5 hours, about 4 hours, or about 3 hours. In some embodiments, Compound A4 is reacted with 2-bromo-1,1-dimethoxyethane in the presence of zinc bromide for about 5-9 hours.

In some embodiments, Step A4 comprises reacting Compound A4 with 2-bromo-1,1-dimethoxyethane in the presence of an acid or a Lewis acid at a temperature below about 100° C. for less than about 24 hours. In some embodiments, Step A4 comprises reacting Compound A4 with 2-bromo-1,1-dimethoxyethane in the presence of an acid or a Lewis acid at a temperature of about 80° C. for about 5-9 hours.

In some embodiments, Step A4 comprises reacting Compound A4 with 2-bromo-1,1-dimethoxyethane in the presence of zinc bromide at a temperature below about 100° C. for less than about 24 hours. In some embodiments, Step A4 comprises reacting Compound A4 with 2-bromo-1,1-dimethoxyethane in the presence of zinc bromide at a temperature of about 80° C. for about 5-9 hours.

In some embodiments, Step A4 may further comprise distilling the reaction mixture. In some embodiments, Step A4 may further comprise distilling the reaction mixture for about 3-4 volumes. In some embodiments, Step A4 may further comprise distilling the reaction mixture and then cooling to a temperature between about 15° C. and about 25° C.

In some embodiments, Step A4 may further comprise adding an aqueous solution of Na₂CO₃. In some embodiments, Step A4 may further comprise slowly adding an aqueous solution of Na₂CO₃. In some embodiments, Step A4 may further comprise adding methyl tert-butyl ether (MTBE). In some embodiments, Step A4 may further comprise adding an aqueous solution of Na₂CO₃ and MTBE. In some embodiments, Step A4 may further comprise adding an aqueous solution of Na₂CO₃ and MTBE and stirring for about 1-2 hours to form a solid.

In some embodiments, Step A4 may further comprise isolating the solid by filtration. In some embodiments, Step A4 may further comprise isolating the solid by filtration and washing the solid with MTBE. In some embodiments, Step A4 may further comprise stirring the filtrate to allow separation of organic and inorganic layers and isolating the upper organic layer. In some embodiments, the upper organic layer is washed with water, decolorized (e.g., with CUNO™) and washed with MTBE to form an MTBE solution.

In some embodiments, Step A4 may further comprise concentrating the MTBE solution. In some embodiments, Step A4 may further comprise solvent swap to heptane and distilling to form a slurry comprising a solid. In some embodiments, Step A4 may further comprise filtering the solid and washing it with an organic solvent. In some embodiments, Step A4 may further comprise filtering the solid and washing it with n-heptane to give crude Compound A5.

In some embodiments, Step A4 may further comprise diluting crude Compound A5 with acetone. In some embodiments, Step A4 may further comprise diluting Compound A5 with water. In some embodiments, Step A4 may further comprise diluting Compound A5 with acetone and water. In some embodiments, Step A4 may further comprise extracting Compound A5. In some embodiments, Step A4 may further comprise extracting Compound A5 by filtration and washing the filter cake with water. In some embodiments, Step A4 may further comprising drying the solid, Compound A5 (e.g., overnight).

In some embodiments, the yield of Compound A5 in Step A4 is at least about 55%, at least about 60%, at least about 65%, at least about 70%, or at least about 75%. In some embodiments, the yield of Compound A5 in Step A4 is at least about 60%.

In some embodiments, the purity of Compound A5 in Step A4 is at least about 70%, at least about 75%, at least about 80%, at least about 85%, at least about 85%, at least about 90%, at least about 95%, at least about 98%, or at least about 99%.

### Step A4 (Alternative Route)

In some embodiments, Step A4 comprises adding ytterbium triflate, Yb(OTf)₃, to Compound A4. In some embodiments, Step A4 comprises adding a solvent to Compound A4. In some embodiments, Step A4 comprises adding a polar protic solvent (e.g., acetonitrile) to Compound A4 and Yb(OTf)₃. In some embodiments, Step A4 comprises adding acetonitrile to Compound A4 and Yb(OTf)₃.

In some embodiments, Step A4 comprises adding 2-bromo-1,1-dimethoxyethane to Compound A4 and Yb(OTf)₃ in acetonitrile. In some embodiments, Step A4 comprises adding 2-bromo-1,1-dimethoxyethane to Compound A4 and Yb(OTf)₃ in acetonitrile at a temperature below about 100° C. In some embodiments, Compound A4 is reacted with 2-bromo-1,1-dimethoxyethane in the presence of Yb(OTf)₃ at a temperature of about 85° C. In some embodiments, Compound A4 is reacted with 2-bromo-1,1-dimethoxyethane in the presence of Yb(OTf)₃ at a temperature of about 55° C., about 60° C., about 65° C., about 70° C., about 75° C., about 80° C., about 85° C., about 90° C., about 95° C., or about 100° C.

In some embodiments, Step A4 comprises reacting Compound A4 with 2-bromo-1,1-dimethoxyethane in the presence of Yb(OTf)₃ for less than about 24 hours. In some embodiments, Compound A4 is reacted with 2-bromo-1,1-dimethoxyethane in the presence of Yb(OTf)₃ for less than about 20 hours, less than about 15 hours, less than about 10 hours, or less than about 9 hours. In some embodiments, Compound A4 is reacted with 2-bromo-1,1-dimethoxyethane in the presence of Yb(OTf)₃ for about 12 hours, about 11 hours, about 10 hours, about 9 hours, about 8 hours, about 7 hours, about 6 hours, about 5 hours, about 4 hours, or about 3 hours. In some embodiments, Compound A4 is reacted with 2-bromo-1,1-dimethoxyethane in the presence of Yb(OTf)₃ for about 5-9 hours.

In some embodiments, Step A4 comprises reacting Compound A4 with 2-bromo-1,1-dimethoxyethane in the presence of Yb(OTf)₃ at a temperature below about 100° C. for less than about 24 hours. In some embodiments, Step A4 comprises reacting Compound A4 with 2-bromo-1,1-dimethoxyethane in the presence of Yb(OTf)₃ at a temperature of about 80° C. for about 5-9 hours.

In some embodiments, Step A4 may further comprise distilling the reaction mixture. In some embodiments, Step A4 may further comprise distilling the reaction mixture for about 2-3 volumes. In some embodiments, Step A4 may further comprise distilling the reaction mixture and then cooling to a temperature between about 15° C. and about 25° C.

In some embodiments, Step A4 may further comprise adding an aqueous solution of NaHCO₃. In some embodiments, Step A4 may further comprise adding an aqueous solution of Na₂HCO₃ and to form a solid.

In some embodiments, Step A4 may further comprise isolating the solid. In some embodiments, Step A4 may further comprise isolating the solid and washing the solid with water and MTBE. In some embodiments, Step A4 may further comprise decolorizing the MTBE layer (e.g., with CUNO™) and washed with water.

In some embodiments, Step A4 may further comprise concentrating the MTBE solution. In some embodiments, Step A4 may further comprise adding heptane to form a solid. In some embodiments, Step A4 may further comprise filtering the solid and washing it with an organic solvent. In some embodiments, Step A4 may further comprise filtering the solid and washing it with n-heptane to give crude Compound A5.

In some embodiments, Step A4 may further comprise diluting crude Compound A5 with acetone. In some embodiments, Step A4 may further comprise diluting Compound A5 with water. In some embodiments, Step A4 may further comprise diluting Compound A5 with acetone and water. In some embodiments, Step A4 may further comprise extracting Compound A5. In some embodiments, Step A4 may further comprise extracting Compound A5 by filtration and washing the filter cake with water. In some embodiments, Step A4 may further comprising drying the solid, Compound A5 (e.g., overnight).

In some embodiments, the yield of Compound A5 in Step A4 is at least about 50%, at least about 55%, at least about 60%, at least about 65%, at least about 70%, or at least about 75%. In some embodiments, the yield of Compound A5 in Step A4 is at least about 55%.

In some embodiments, the purity of Compound A5 in Step A4 is at least about 70%, at least about 75%, at least about 80%, at least about 85%, at least about 85%, at least about 90%, at least about 95%, at least about 98%, or at least about 99%.

In some embodiments, the Lewis acid is selected from the group consisting of zinc bromide, ytterbium triflate, iron chloride, scandium triflate, copper triflate, zinc triflate, barium triflate, samarium triflate, indium triflate, cerium triflate, tin triflate, iron triflate, calcium triflate, aluminum triflate, scandium triflate, yttrium triflate, zinc acetate, zinc chloride, cobalt chloride and zinc iodide.

In some embodiments, the acid includes but are not limited to p-toluene sulfonic acid (p-TSA), pyridium p-toluene sulfonate (PPTS), acetic acid, trifluoroacetic acid (TFA), methane sulfonic acid (MSA), trifluoromethanesulfonic acid (TFA), camphor sulfonic acid (CSA), hydrochloric acid, hydrobromic acid.

In some embodiments, Step A4 forms Compound A5 and Compound SP1:

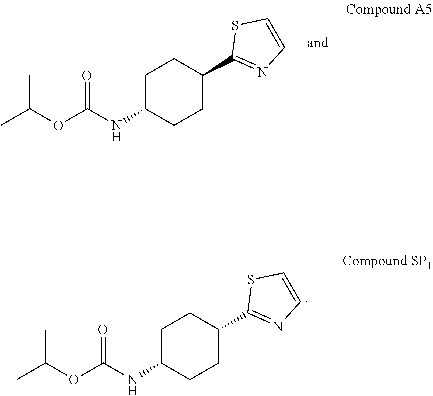 [SMILES: CC(C)OC(=O)N[C@H]1CC[C@H](c2nccs2)CC1 ; CC(C)OC(=O)N[C@H]1CC[C@@H](c2nccs2)CC1]

In some embodiments, the acid/Lewis acid and solvent for Step A4 can be seen in Table 1.

TABLE 1
Acid/Lewis acid and solvent for Step A4

| | | Combined | |
|---|---|---|---|
| | | Compound | Compound |
| Acid/Lewis | | A5 & Compound | A5:Compound |
| Acid | Conditions | SP₁ Formed | SP₁ |
| Yb(OTf)₃•H₂O | | 79 | 92:8 |
| Y(OTf)₃ | | 77 | 88:12 |
| Sc(OTf)₃ | | 49 | 92:8 |
| Zn(OTf)₂ | | 80 | 65:35 |
| FeCl₂ | | <5 | Not measured |
| FeCl₂ | Reagent = | 80 | 80:20 |
| | Bromoacetaldehyde | | |
| CoCl₂ | | 61 | 79:21 |
| CeCl₃ | | 80 | 50:50 |
| YbCl₃•6H₂O | | | |
| TiCl₄ | | 36 | 65:35 |
| BF₃•OEt₂ | | 13 | 65:35 |
| p-TSA | | 8 | 65:35 |
| p-TSA | Reagent = | 80 | 50:50 |
| | Bromoacetaldehyde | | |
| Acetic acid | Reagent = | 45 | 75:25 |
| | Bromoacetaldehyde | | |
| CSA | Reagent = | 87 | 75:25 |
| | Bromoacetaldehyde | | |
| CSA | | 29 | 58:42 |
| Aq HCl | | 27 | 57:43 |
| HCl | | <5% | Not measured |
| (anhydrous) | | | |

Note:
Unless stated all reactions use MeCN as solvent, 2-Bromo-1,1-dimethoxyethane as reagent with a reaction temperature of 80° C.

Embodiment 7: In some aspects, the method of Embodiment 6 comprises cyclization of Compound A4 in the presence of an acid or a Lewis acid selected from ZnBr₂, Yb(OTf)₃·H₂O, Y(OTf)₃, Sc(OTf)₃, Zn(OTf)₂, FeCl₂, CoCl₂, p-toluene sulfonic acid, and camphor sulfonic acid, or a combination thereof.

Embodiment 8: In some aspects, the method of Embodiment 6 or 7 comprises cyclization of Compound A4 in the presence of 2-bromo-1,1-dimethoxy-ethane; 2-bromo-1,1-diethoxyethane; 2-bromoacetaldehyde; or 2-chloroacetaldehyde; or a combination thereof.

Embodiment 9: In some aspects, in any one of Embodiments 6-8, Step A4 is as follows:

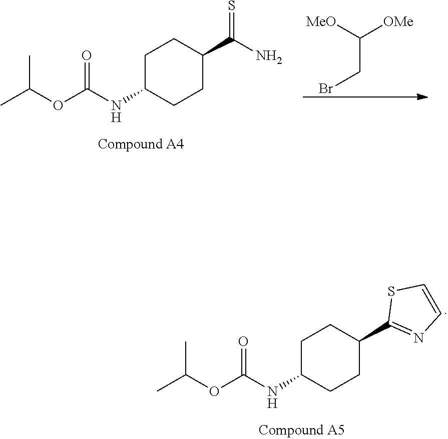 [SMILES: CC(C)OC(=O)N[C@H]1CC[C@H](C(N)=S)CC1 ; COC(CBr)OC ; CC(C)OC(=O)N[C@H]1CC[C@H](c2nccs2)CC1]

Embodiment 10: In some aspects, the method of any one of Embodiments 6-9 comprises cyclization of Compound A4 in the presence of ZnBr₂, Yb(OTf)₃·H₂O, Y(OTf)₃, or Sc(OTf)₃, or a combination thereof.

Embodiment 11: In some aspects, the method of any one of Embodiments 6-10 comprises cyclization of Compound A4 in the presence of ZnBr₂.

Embodiment 12: In some aspects, in any one of Embodiments 6-11, Step A4 forms Compound A5 and Compound SP1:

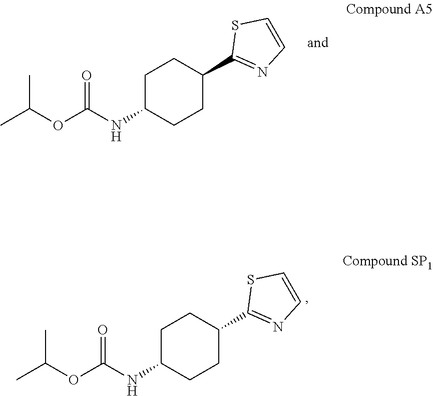 [SMILES: CC(C)OC(=O)N[C@H]1CC[C@H](c2nccs2)CC1 ; CC(C)OC(=O)N[C@H]1CC[C@@H](c2nccs2)CC1]

wherein the wt/wt ratio in the amount between Compound A5 and Compound SP1 is at least about 50:50, at least about 55:45, at least about 60:40, at least about 65:35, at least about 70:30, at least about 75:25, at least about 80:20, at least about 85:15, at least about 90:10, at least about 92:8, or at least about 95:5.

Embodiment 13: In some aspects, in any one of Embodiments 6-12, Step A4 forms Compound A5 and Compound SP1:

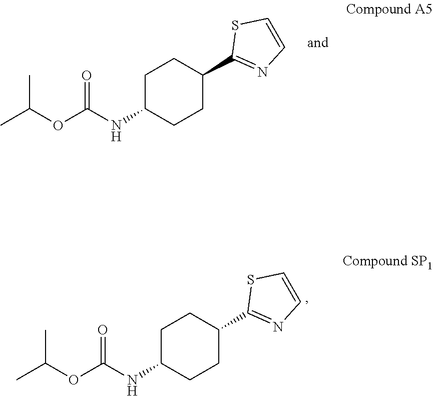 [SMILES: CC(C)OC(=O)N[C@H]1CC[C@H](c2nccs2)CC1 ; CC(C)OC(=O)N[C@H]1CC[C@@H](c2nccs2)CC1]

wherein the total yield of Compound A5 and Compound SP1 is at least about 40%, at least about 50%, at least about 55%, at least about 60%, at least about 65%, at least about 70%, at least about 75%, at least about 80%, at least about 85%, or at least about 90%.

Embodiment 14: In some aspects, the method of any one of Embodiments 6-13 further comprises one or more steps of Step A1, Step A2, and Step A3:
Step A1: reacting Compound A1:

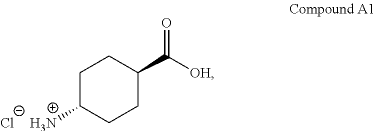

with isopropyl chloroformate to form Compound A2:

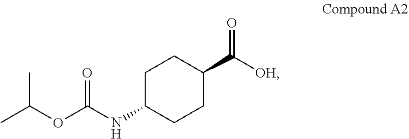 [SMILES: CC(C)OC(=O)N[C@H]1CC[C@H](C(=O)O)CC1]

Step A2: reductive amination of Compound A2 to form Compound A3:

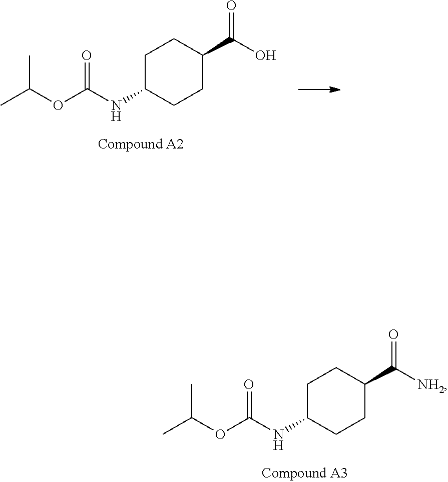 [SMILES: CC(C)OC(=O)N[C@H]1CC[C@H](C(=O)O)CC1 ; CC(C)OC(=O)N[C@H]1CC[C@H](C(N)=O)CC1]

and
Step A3: converting Compound A3 to Compound A4:

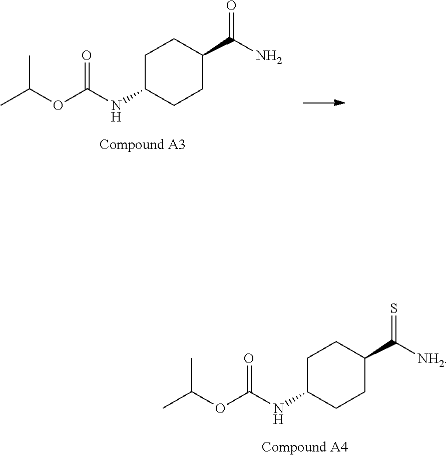 [SMILES: CC(C)OC(=O)N[C@H]1CC[C@H](C(N)=O)CC1 ; CC(C)OC(=O)N[C@H]1CC[C@H](C(N)=S)CC1]

### Step A1

In some embodiments, the present application provides the method of making Compound A2 or a pharmaceutically acceptable salt or solvate thereof. In some embodiments, the present application provides the method of making Compound A2.

In some embodiments, Compound A2 or a pharmaceutically acceptable salt or solvate thereof is prepared by a method disclosed herein. In some embodiments, Compound A2 is prepared by a method disclosed herein.

In some embodiments, Compound A2 is prepared by adding isopropyl chloroformate to Compound A1.

In some embodiments, the method of the present application comprises:
Step A1: reacting Compound A1:

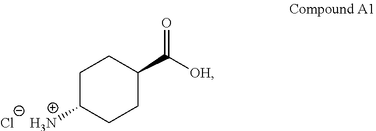

with isopropyl chloroformate to form Compound A2:

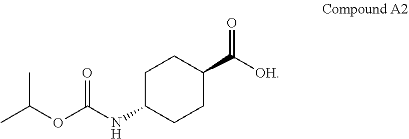 [SMILES: CC(C)OC(=O)N[C@H]1CC[C@H](C(=O)O)CC1]

In some embodiments, Step A1 comprises adding isopropyl chloroformate to Compound A1.

In some embodiments, Step A1 comprises adding a solution of isopropyl chloroformate to Compound A1. In some embodiments, Step A1 comprises reacting Compound A1 with a solution of isopropyl chloroformate in the presence of a base (e.g., sodium hydroxide). In some embodiments, Step A1 comprises adding Compound A1 to a base (e.g., a solution of sodium hydroxide) to form a mixture and adding a solution of isopropyl chloroformate to the mixture.

In some embodiments, Step A1 comprises reacting Compound A1 with a solution of isopropyl chloroformate at a temperature below about 30° C. In some embodiments, Compound 1 is reacted with a solution of isopropyl chloroformate at about room temperature. In some embodiments, Compound A1 is reacted with 1M sodium hydroxide followed by the addition of a solution of isopropyl chloroformate at a temperature below about 30° C. In some embodiments, Compound A1 is reacted with 1M sodium hydroxide followed by the addition of a solution of isopropyl chloroformate at about room temperature.

In some embodiments, Step A1 comprises reacting Compound A1 with a solution of isopropyl chloroformate for less than about 10 hours. In some embodiments, Compound A1 is reacted with a solution of isopropyl chloroformate for less than about 6 hours. In some embodiments, Compound A1 is reacted with a solution of isopropyl chloroformate for about 4 to about 6 hours.

In some embodiments, Step A1 may further comprise cooling. In some embodiments, Step A1 may further comprise adding 6M aq HCl and stirring (e.g., for about 6 hours).

In some embodiments, Step A1 may further comprise stirring (e.g., for about 6 hours). In some embodiments, a precipitate is formed.

In some embodiments, the precipitate is filtered (e.g., with a Buchner funnel). In some embodiments, Step A1 may further comprise washing the precipitate. In some embodiments,

Step A1 may further comprise washing the precipitate with water. In some embodiments, Step A1 may further comprise washing the precipitate, Compound A2, with water. In some embodiments, Step A1 may further comprise drying Compound A2.

In some embodiments, Step A1 may further comprising drying the Compound A2 for about 5 days.

In some embodiments, Step A1 may further comprising drying the Compound A2 for about 5 days at a temperature above room temperature (e.g., about 45-65° C.).

In some embodiments, the yield of Compound A2 in Step A1 is at least about 70%, at least about 75%, at least about 80%, at least about 85%, at least about 85%, at least about 90%, or at least about 95%. In some embodiments, the yield of Compound A2 in Step A1 is at least about 85%.

In some embodiments, the purity of Compound A2 in Step A1 is at least about 70%, at least about 75%, at least about 80%, at least about 85%, at least about 85%, at least about 90%, at least about 95%, at least about 98%, or at least about 99%. In some embodiments, the purity of Compound A2 in Step A1 is at least about 98%.

### Step A1 (Alternative Route)

In some embodiments, Step A1 comprises forming Compound A2 using phase transfer condition. In some embodiments, Step A1 comprises adding Compound A1 to a base (e.g., sodium hydroxide). In some embodiments, Step A1 comprises adding Compound A1 to a sodium hydroxide solution. In some embodiments, Step A1 comprises adding Compound A1 to a sodium hydroxide solution and then adding tetrabutylammonium bromide (TBAB) to form a solution. In some embodiments, the solution is stirred at a temperature below room temperature. In some embodiments, the solution is stirred at a temperature between about 0° C. and 20° C.

In some embodiments, Step A1 further comprises adding a solution of isopropyl chloroformate in toluene to form a second solution. In some embodiments, Step A1 comprises stirring the second solution for less than about 40 hours. In some embodiments, the second solution is stirred for less than about 36 hours, less than about 30 hours, less than about 24 hours, or less than about 20 hours. In some embodiments, the second solution is stirred for about 24 hours. In some embodiments, Step A1 comprises reacting Compound A1 with a solution of isopropyl chloroformate in toluene at a temperature below about 30° C. for less than about 40 hours. In some embodiments, Step A1 comprises reacting Compound A1 with a solution of isopropyl chloroformate in toluene at a temperature between about 0° C. and room temperature for less than about 36 hours, less than about 30 hours, less than about 24 hours, or less than about 20 hours. In some embodiments, Step A1 comprises reacting Compound A1 with a solution of isopropyl chloroformate in toluene at a temperature between about 15° C. and about 25° C. for about 24 hours. In some embodiments, Step A1 comprises reacting Compound A1 with a solution of isopropyl chloroformate in toluene in the presence of a base (e.g., sodium hydroxide) at a temperature between about 0° C. and about room temperature for about 24 hours. In some embodiments, Step A1 comprises reacting Compound A1 with a solution of isopropyl chloroformate in toluene in the presence of a base (e.g., sodium hydroxide) at a temperature between about 15° C. and about 25° C. for about 24 hours.

In some embodiments, Step A1 may further comprise extracting the reaction with a solvent. In some embodiments, Step A1 may further comprise extracting the reaction with a polar solvent (e.g., ethyl acetate). In some embodiments, Step A1 may further comprise extracting the reaction with ethyl acetate.

In some embodiments, Step A1 may further comprise adding an acid to the aqueous layer. In some embodiments, Step A1 may further comprise adding 6M aqueous HCl solution to the aqueous layer. In some embodiments, the 6M aqueous HCl solution is added dropwise. In some embodiments, the solution is stirred (e.g., for about 6 hours). In some embodiments, HCl is added gradually.

In some embodiments, Step A1 may further comprise cooling (e.g., to a temperature of about 0-10° C.). In some embodiments, a precipitate, Compound A2, is formed. In some embodiments, the precipitate is filtered (e.g., with a Buchner funnel). In some embodiments, Step A1 may further comprise washing the precipitate, Compound A2. In some embodiments, Step A1 may further comprise washing the precipitate, Compound A2, with water.

In some embodiments, Step A1 may further comprise drying Compound A2. In some embodiments, Step A1 may further comprising drying Compound A2 at a temperature above room temperature. In some embodiments, Step A1 may further comprising drying Compound A2 at a temperature above room temperature for about 1 day. In some embodiments, Step A1 may further comprising drying Compound A2 at a temperature from about 45-55° C. In some embodiments, Step A1 may further comprising drying Compound A2 at a temperature from about 45-55° C. for about 1 day.

In some embodiments, the yield of Compound A2 in Step A1 is at least about 70%, at least about 75%, at least about 80%, at least about 85%, at least about 85%, at least about 90%, or at least about 95%. In some embodiments, the yield of Compound A2 in Step A1 is at least about 80%.

In some embodiments, the purity of Compound A2 in Step A1 is at least about 70%, at least about 75%, at least about 80%, at least about 85%, at least about 85%, at least about 90%, at least about 95%, at least about 98%, or at least about 99%.

### Step A2

In some embodiments, the present application provides the method of making Compound A3 or a pharmaceutically acceptable salt or solvate thereof. In some embodiments, the present application provides the method of making Compound A3.

In some embodiments, Compound A3 or a pharmaceutically acceptable salt or solvate thereof is prepared by a method disclosed herein. In some embodiments, Compound A3 is prepared by a method disclosed herein.

In some embodiments, Compound A3 is prepared by adding ethyl chloroformate and ammonium hydroxide to Compound A2.

In some embodiments, the method of the present application comprises Step A2: reacting Compound A2 with 1,1′-carbonyldiimidazole (CDI) and ammonium hydroxide to form Compound A3.

In some embodiments, Step A2 comprises adding an aprotic solvent (e.g., ethyl acetate) to Compound A2. In some embodiments, Step A2 comprises adding ethyl acetate to Compound A2.

In some embodiments, Step A2 comprises adding CDI and ammonium hydroxide to Compound A2 and the aprotic solvent (e.g., ethyl acetate).

In some embodiments, Step A2 comprises stirring Compound A2, the aprotic solvent (e.g., ethyl acetate), CDI and ammonium hydroxide at about room temperature.

In some embodiments, Step A2 comprises stirring Compound A2, the aprotic solvent (e.g., ethyl acetate), CDI and ammonium hydroxide at a temperature between about 15° C. and about −25° C. In some embodiments, Step A2 comprises stirring Compound A2, the aprotic solvent (e.g., ethyl acetate), CDI and ammonium hydroxide at a temperature between about 15° C. and about −25° C. for about 12 hours, about 10 hours, about 8 hours, about 6 hours, about 4 hours, or about 2 hours to form a resultant solid, Compound A3.

In some embodiments, Step A2 comprises filtering the resultant solid, Compound A3. In some embodiments, Step A2 comprises filtering and collecting the resultant solid (i.e., Compound A)₃. In some embodiments, the filter cake is washed. In some embodiments, the filter cake is washed with water. In some embodiments, the filter cake is mixed with MeCN and water to form a slurry. In some embodiments, the slurry is stirred at a temperature below room temperature. In some embodiments, the slurry solution is stirred at a temperature between about 15° C. and about −25° C. In some embodiments, the slurry solution is stirred at a temperature between about 15° C. and about −25° C. for about six hours. In some embodiments, the slurry solution is stirred at a temperature between about 15° C. and about −25° C. for about six hours and filtered. In some embodiments, the resultant precipitate is filtered and collected. In some embodiments, the resultant precipitate is filtered, the filter cake washed with MeCN and Compound A3 is collected. In some embodiments, Step A2 may further comprises drying the solid, Compound A3.

In some embodiments, the yield of Compound A3 in Step A2 is at least about 60%, at least about 65%, at least about 70%, at least about 75%, at least about 80%, at least about 85%, at least about 85%, at least about 90%, or at least about 95%. In some embodiments, the yield of Compound A3 in Step A2 is at least about 90%.

In some embodiments, the purity of Compound A3 in Step A2 is at least about 70%, at least about 75%, at least about 80%, at least about 85%, at least about 85%, at least about 90%, at least about 95%, at least about 98%, or at least about 99%. In some embodiments, the purity of Compound A3 in Step A2 is at least about 98%.

### Step A2 (Alternative Route)

In some embodiments, Step A2 comprises adding triethyl amine to Compound A2. In some embodiments, Step A2 comprises adding an aprotic solvent (e.g., THF) to Compound A2. In some embodiments, Step A2 comprises adding THF to Compound A2. In some embodiments, Step A2 comprises adding an amine (e.g., triethylamine) to Compound A2 in THF. In some embodiments, Step A2 comprises adding triethylamine to Compound A2 in THF.

In some embodiments, Step A2 comprising reacting Compound A2 with triethylamine in THF with iso-propyl chloroformate. In some embodiments, Step A2 comprising reacting Compound A2 with triethylamine in THF with a chloroformate.

In some embodiments, Step A2 comprises reacting Compound A2 with iso-propyl chloroformate at a temperature below about 0° C. In some embodiments, Compound A2 is reacted with iso-propyl chloroformate below about −10° C. In some embodiments, Compound A2 is reacted with iso-propyl chloroformate at a temperature of about −15° C.

In some embodiments, Step A2 comprising reacting Compound A2 with triethylamine in THF with a chloroformate below about 0° C. In some embodiments, Step A2 comprising reacting Compound A2 with triethylamine in THF with a chloroformate at a temperature of about −15° C.

In some embodiments, Compound A2 is reacted with iso-propyl chloroformate at a temperature of about −15° C. followed by the addition of a base. In some embodiments, Compound A2 is reacted with iso-propyl chloroformate followed by the addition of ammonium hydroxide (e.g., about 30% aqueous). In some embodiments, Compound A2 is reacted with iso-propyl chloroformate followed by the addition of ammonium hydroxide (e.g., about 30% aqueous) at a temperature below room temperature. In some embodiments, Compound A2 is reacted with iso-propyl chloroformate followed by the addition of ammonium hydroxide (e.g., about 30% aqueous) at a temperature between about 15° C. and about 25° C. In some embodiments, Compound A2 is reacted with iso-propyl chloroformate followed by the addition of ammonium hydroxide (e.g., about 30% aqueous) at a temperature of about 10° C., about 20° C., or about 25° C. In some embodiments, Step A2 comprises reacting Compound A2 with iso-propyl chloroformate and ammonium hydroxide for less than about 100 hours. In some embodiments, Compound A2 is reacted with iso-propyl chloroformate and ammonium hydroxide for less than about 72 hours. In some embodiments, Compound A2 is reacted with iso-propyl chloroformate and ammonium hydroxide for about 48-72 hours.

In some embodiments, Step A2 may further comprise concentrating the solution. In some embodiments, Step A2 may further comprise concentrating the solution under vacuum and filtering the resultant solid, Compound A3.

In some embodiments, Step A2 may further comprise washing the resultant solid. In some embodiments, Step A2 may further comprise washing the solid with water. In some embodiments, Step A2 may further comprise washing the solid with isopropanol. In some embodiments, Step A2 may further comprise washing the solid with heptane. In some embodiments, Step A2 may further comprise washing the solid with an isopropanol/heptane mixture (e.g., 10% isopropanol/heptane).

In some embodiments, Step A2 may further comprising drying the solid, Compound A3, overnight.

In some embodiments, the yield of Compound A3 in Step A2 is at least about 70%, at least about 75%, at least about 80%, at least about 85%, at least about 85%, at least about 90%, or at least about 95%. In some embodiments, the yield of Compound A3 in Step A2 is at least about 70%.

In some embodiments, the purity of Compound A3 in Step A2 is at least about 70%, at least about 75%, at least about 80%, at least about 85%, at least about 85%, at least about 90%, at least about 95%, at least about 98%, or at least about 99%.

### Step A3

In some embodiments, the present application provides the method of making Compound A4 or a pharmaceutically acceptable salt or solvate thereof. In some embodiments, the present application provides the method of making Compound A4.

In some embodiments, Compound A4 or a pharmaceutically acceptable salt or solvate thereof is prepared by a method disclosed herein. In some embodiments, Compound A4 is prepared by a method disclosed herein.

In some embodiments, Compound A4 is prepared by adding a thionating agent (e.g., Lawesson's reagent) to Compound A3.

In some embodiments, the method of the present application comprises Step A3: reacting Compound A3 with Lawesson's reagent to form Compound A4.

In some embodiments, Step A3 comprises adding Lawesson's reagent to Compound A3. In some embodiments, Step A3 comprises adding a solvent to Compound A3. In some embodiments, Step A3 comprises adding an aprotic solvent (e.g., 2-MeTHF) to Compound A3. In some embodiments, Step A3 comprises adding 2-MeTHF to Compound A3. In some embodiments, Step A3 comprises adding Lawesson's reagent to Compound A3 in the aprotic solvent. In some embodiments, Step A3 comprises adding Lawesson's reagent to Compound A3 in 2-MeTHF.

In some embodiments, Step A3 comprises reacting Compound A3 with Lawesson's reagent at a temperature below about 50° C. In some embodiments, Compound A3 is reacted with Lawesson's reagent at about room temperature. In some embodiments, Compound A3 is reacted with Lawesson's reagent at a temperature between about 20° C. and 30° C. In some embodiments, Compound A3 is reacted with Lawesson's reagent at a temperature of about 15° C., about 20° C., about 25° C., about 30° C., about 35° C., or about 40° C.

In some embodiments, Step A3 comprises reacting Compound A3 with Lawesson's reagent for less than about 40 hours. In some embodiments, Compound A3 is reacted with Lawesson's reagent for less than about 36 hours, less than about 30 hours, less than about 24 hours, or less than about 20 hours. In some embodiments, Compound A3 is reacted with Lawesson's reagent for about 24 hours.

In some embodiments, Step A3 comprises adding of Na₂CO₃. In some embodiments, Step A3 comprises, after Na₂CO₃ addition, the reaction mixture is stirred. In some embodiments, Step A3 comprises, after Na₂CO₃ addition, the reaction mixture is stirred at a temperature below about 30° C. In some embodiments, Step A3 comprises, after Na₂CO₃ addition, the reaction mixture is stirred at about room temperature. In some embodiments, Step A3 comprises, after Na₂CO₃ addition, the reaction mixture is stirred at a temperature between about 20° C. and 30° C. and layers allowed to separate. In some embodiments, Step A3 comprises, after Na₂CO₃ addition, the reaction mixture is stirred a temperature between about 20° C. and 30° C. and organic layer washed with water.

In some embodiments, the organic solution of Step A3 is concentrated. In some embodiments, pure Compound A4 is added to the dissolved solution of Compound A. In some embodiments, pure Compound A4 is added to dissolved solution of Compound A4 at a temperature of about 35-45° C. In some embodiments, pure Compound A4 is added to dissolved solution of Compound A4 at a temperature of about 35-45° C. and heptane added to form a resultant precipitate, Compound A4. In some embodiments, the resultant precipitate is filtered. In some embodiments, the resultant precipitate is filtered and collected. In some embodiments, the resultant precipitate is filtered, the filter cake washed with heptane and Compound A4 is collected. In some embodiments, Step A3 may further comprises drying the solid, Compound A4.

In some embodiments, the yield of Compound A4 in Step A3 is at least about 60%, at least about 65%, at least about 70%, at least about 75%, at least about 80%, at least about 85%, at least about 85%, at least about 90%, or at least about 95%. In some embodiments, the yield of Compound A4 in Step A3 is at least about 70%.

In some embodiments, the purity of Compound A4 in Step A3 is at least about 70%, at least about 75%, at least about 80%, at least about 85%, at least about 85%, at least about 90%, at least about 95%, at least about 98%, or at least about 99%. In some embodiments, the purity of Compound A4 in Step A3 is at least about 90%.

Embodiment 15: In some aspects, the method of any one of Embodiments 1-5 further comprises Step A4′:
Step A4′: hydrogenation of Compound Aₐₜ:

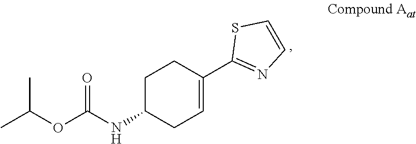 [SMILES: CC(C)OC(=O)N[C@H]1CC=C(c2nccs2)CC1 ; ","]

to form Compound A5:

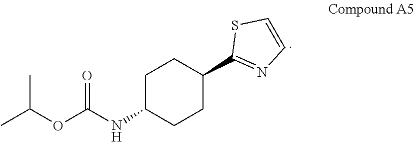 [SMILES: CC(C)OC(=O)N[C@H]1CC[C@H](c2nccs2)CC1 ; "."]

### Step A4′ (Alternative Route to Form Compound A5)

In some embodiments, Compound A5 can be prepared from Compound Aₐₜ:

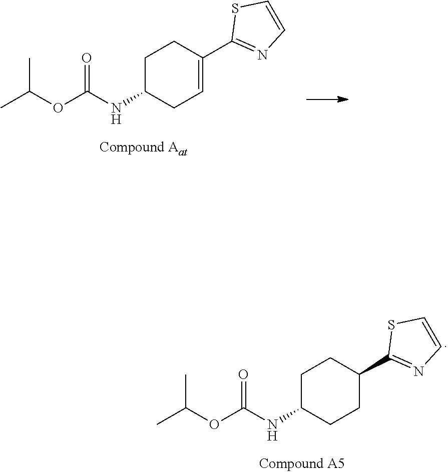 [SMILES: CC(C)OC(=O)N[C@H]1CC=C(c2nccs2)CC1 ; CC(C)OC(=O)N[C@H]1CC[C@H](c2nccs2)CC1]

In some embodiments, Compound A5 can be prepared from Compound Aₐₜ in the presence of a catalyst (e.g., Pd/C, (PPh₃)₃RhCl (Wilkinson's Catalyst), Pd(OH)₂, PtO₂, and [C₈H₁₂IrP(C₆H₁₁)₃C₅H₅N]PF₆ (Crabtree's catalyst)). In some embodiments, the yield of Compound A5 in Step A4′ is at least about 70%, at least about 75%, at least about 80%, at least about 85%, at least about 85%, at least about 90%, or at least about 95%.

In some embodiments, the purity of Compound A5 in Step A4′ is at least about 70%, at least about 75%, at least about 80%, at least about 85%, at least about 85%, at least about 90%, at least about 95%, at least about 98%, or at least about 99%.

Embodiment 16: In some aspects, the method of any one of Embodiments 1-15 further comprises Step A5:
Step A5: reacting Compound A5:

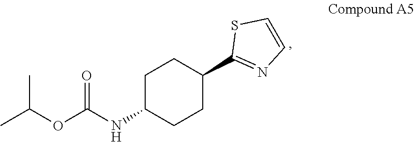 [SMILES: CC(C)OC(=O)N[C@H]1CC[C@H](c2nccs2)CC1 ; ","]

with a halogenating agent to form Compound X:

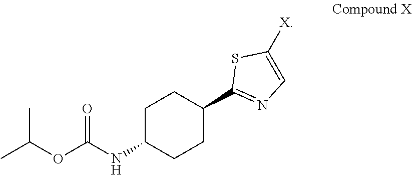

### Step A5

In some embodiments, the present application provides the method of making Compound X or a pharmaceutically acceptable salt or solvate thereof. In some embodiments, the present application provides the method of making Compound X.

In some embodiments, Compound X or a pharmaceutically acceptable salt or solvate thereof is prepared by a method disclosed herein. In some embodiments, Compound X is prepared by a method disclosed herein.

In some embodiments, Compound X is prepared by adding a halogenating agent to Compound A5. In some embodiments, the halogenating agent of Step A5 is bromine, hydrogen bromide, or carbon tetrabromide.

In some embodiments, the method of making Compound X comprises Step A1, Step A2, Step A3, Step A4, and Step A5. In some embodiments, the method of making Compound X comprises Step A1. In some embodiments, the method of making Compound X comprises Step A2. In some embodiments, the method of making Compound X comprises Step A3. In some embodiments, the method of making Compound X comprises Step A4. In some embodiments, the method of making Compound X comprises Step A5.

In some embodiments, the method of making Compound X comprises Step A1, Step A2, Step A3, Step A4′, and Step A5. In some embodiments, the method of making Compound X comprises Step A1. In some embodiments, the method of making Compound X comprises Step A2. In some embodiments, the method of making Compound X comprises Step A3. In some embodiments, the method of making Compound X comprises Step A4′. In some embodiments, the method of making Compound X comprises Step A5.

In some embodiments, the method of the present application comprises Step A5: reacting Compound A5 with N-bromosuccinimide, to form Compound X.

In some embodiments, Step A5 comprises adding N-bromosuccinimide (NBS) to Compound A5.

In some embodiments, Step A5 comprises adding solvent to Compound A5. In some embodiments, Step A5 comprises adding a polar aprotic solvent to Compound A5. In some embodiments, Step A5 comprises adding DMF to Compound A5. In some embodiments, Step A5 comprises adding N-bromosuccinimide to Compound A5.

In some embodiments, Step A5 comprises adding N-bromosuccinimide to Compound A5 in DMF. In some embodiments, Step A5 comprises reacting Compound A5 with N-bromosuccinimide at a temperature below about 50° C. In some embodiments, Compound A5 is reacted with N-bromosuccinimide at about room temperature.

In some embodiments, Compound A5 is reacted with N-bromosuccinimide at a temperature of about 15° C., about 20° C., about 25° C., about 30° C., about 35° C., about 40° C., about 45° C., about 50° C., about 55° C., or about 60° C.

In some embodiments, Step A5 comprises reacting Compound A5 with N-bromosuccinimide for less than about 40 hours. In some embodiments, Compound A5 is reacted with N-bromosuccinimide for less than about 36 hours, less than about 30 hours, less than about 24 hours, or less than about 20 hours. In some embodiments, Compound A5 is reacted with N-bromosuccinimide for about 20 hours, about 19 hours, about 18 hours, about 17 hours, about 16 hours, or about 15 hours. In some embodiments, Compound A5 is reacted with N-bromosuccinimide for about 16 hours.

In some embodiments, Step A5 comprises reacting Compound A5 with N-bromosuccinimide at a temperature below about 50° C. for less than about 40 hours. In some embodiments, Step A5 comprises reacting Compound A5 with N-bromosuccinimide at a temperature of about room temperature for less than about 36 hours, less than about 30 hours, less than about 24 hours, or less than about 20 hours. In some embodiments, Step A5 comprises reacting Compound A5 with N-bromosuccinimide at a temperature of about room temperature for about 16 hours. In some embodiments, Step A5 further comprises adding water to the reaction after about 16 hours. In some embodiments, the water is added dropwise and temperature is maintained less than about 35° C.

In some embodiments, Step A5 may further comprise filtering the solution. In some embodiments, Step A5 may further comprise filtering the solution to collect a solid. In some embodiments, the solid of Step A5 is Compound X. In some embodiments, Step A5 may further comprise washing Compound X. In some embodiments, Compound X is washed with water. In some embodiments, Compound X is washed with heptane. In some embodiments, Compound X is washed with water and heptane. In some embodiments, Step A5 may further comprising drying the solid, Compound X (e.g., overnight).

In some embodiments, Compound X can be recrystallized. In some embodiments, Compound X can be recrystallized in hot MTBE. In some embodiments, Compound X can be recrystallized. In some embodiments, Compound X can be recrystallized in hot MTBE and collecting the solid. In some embodiments, Compound X can be recrystallized in hot MTBE and collecting the solid (Compound X), washing the solid with water and heptane and drying the solid (e.g., overnight).

In some embodiments, the yield of Compound X in Step A5 is at least about 70%, at least about 75%, at least about 80%, at least about 85%, at least about 85%, at least about 90%, or at least about 95%. In some embodiments, the yield of Compound X in Step A5 is at least about 80%.

In some embodiments, the purity of Compound X in Step A5 is at least about 70%, at least about 75%, at least about 80%, at least about 85%, at least about 85%, at least about 90%, at least about 95%, at least about 98%, or at least about 99%. In some embodiments, the purity of Compound X in Step A5 is at least about 99%.

In some embodiments, Compound A5 is reacted with N-iodosucinimde or N-chlorosuccinimide in DMF to provide the corresponding iodo- and chlor-derivatives which are also expected to react with boronic acids.

Embodiment 17: In some aspects, the method of any one of Embodiments 1-5 further comprises Step A1, Step A2, Step A3, Step A4, and Step A5:
Step A1: reacting Compound A1:

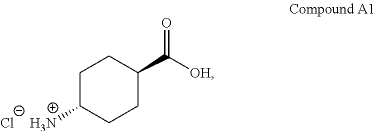

with isopropyl chloroformate to form Compound A2:

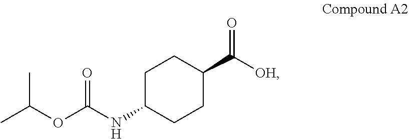 [SMILES: CC(C)OC(=O)N[C@H]1CC[C@H](C(=O)O)CC1]

Step A2: reductive amination of Compound A2 to form Compound A3:

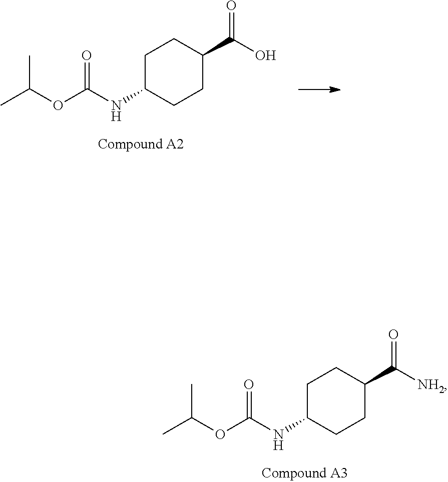 [SMILES: CC(C)OC(=O)N[C@H]1CC[C@H](C(=O)O)CC1 ; CC(C)OC(=O)N[C@H]1CC[C@H](C(N)=O)CC1]

Step A3: converting Compound A3 to Compound A4:

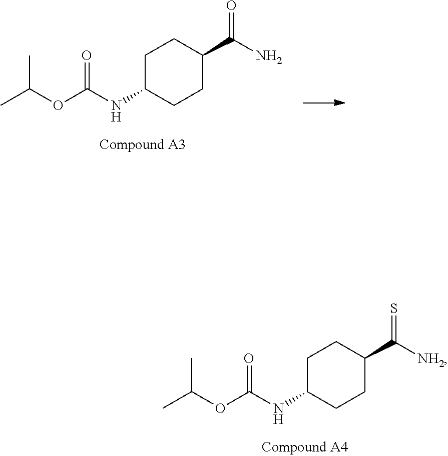 [SMILES: CC(C)OC(=O)N[C@H]1CC[C@H](C(N)=O)CC1 ; CC(C)OC(=O)N[C@H]1CC[C@H](C(N)=S)CC1]

Step A4: cyclization of Compound A4:

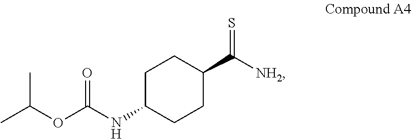 [SMILES: CC(C)OC(=O)N[C@H]1CC[C@H](C(N)=S)CC1]

to form Compound A5:

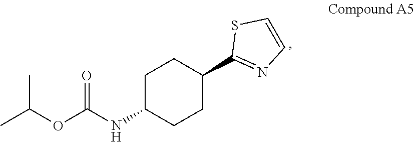 [SMILES: CC(C)OC(=O)N[C@H]1CC[C@H](c2nccs2)CC1 ; ","]

and
Step A5: reacting Compound A5 with a halogenating agent to form Compound X:

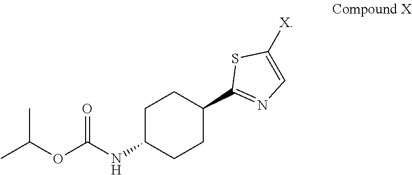

In some embodiments, the method of making Compound X comprises:
Step 1: reacting Compound A1 with isopropyl chloroformate to form Compound A2;
Step 2: contacting a base, a carboxylic acid activator, and a solvent with Compound A2 to form Compound A3;
Step 3: contacting a thionating agent with Compound A3 to form Compound A4;
Step 4: contacting a cyclization agent with Compound A4 to form Compound A5; and
Step 5: contacting a halogenating agent with Compound A5 to form Compound X.

In some embodiments, the method of making Compound X comprises:
Step 1: reacting Compound A1 with isopropyl chloroformate to form Compound A2;
Step 2: contacting a base, a carboxylic acid activator, and a solvent with Compound A2 to form Compound A3;
Step 3: contacting a thionating agent with Compound A3 to form Compound A4;
Step 4′: contacting a hydrogenating agent with Compound Aₐₜ to form Compound A5; and
Step 5: contacting a halogenating agent with Compound A5 to form Compound X.

Embodiment 18: In some aspects, in Embodiment 16 or 17, the halogenating agent is a brominating agent.

Embodiment 19: In some aspects, the method of any one of Embodiments 1-18 further comprises one or more steps of Step B1, Step B2, Step B3, Step B4, and Step B5:
Step B1: converting Compound B1 to Compound B2:

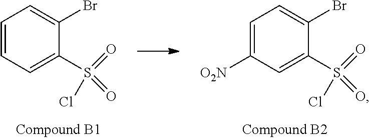 [SMILES: O=S(=O)(Cl)c1ccccc1Br ; O=[N+]([O-])c1ccc(Br)c(S(=O)(=O)Cl)c1]

Step B2: converting of Compound B2 to form Compound B3:

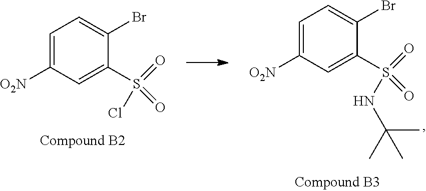 [SMILES: O=[N+]([O-])c1ccc(Br)c(S(=O)(=O)Cl)c1 ; CC(C)(C)NS(=O)(=O)c1cc([N+](=O)[O-])ccc1Br]

Step B3: reducing Compound B3 to Compound B4:

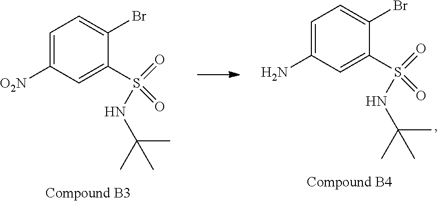 [SMILES: CC(C)(C)NS(=O)(=O)c1cc([N+](=O)[O-])ccc1Br ; CC(C)(C)NS(=O)(=O)c1cc(N)ccc1Br]

Step B4: converting of Compound B4 to form Compound B5:

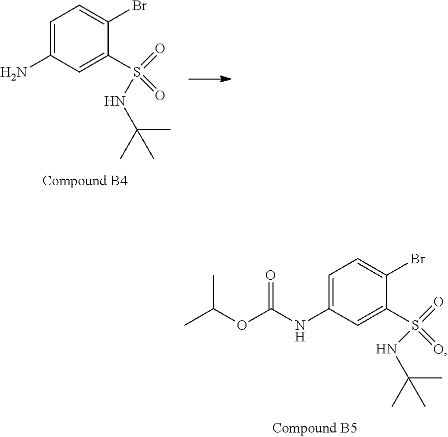 [SMILES: CC(C)(C)NS(=O)(=O)c1cc(N)ccc1Br ; CC(C)OC(=O)Nc1ccc(Br)c(S(=O)(=O)NC(C)(C)C)c1]

and
Step B5: converting of Compound B5 to form Compound Y:

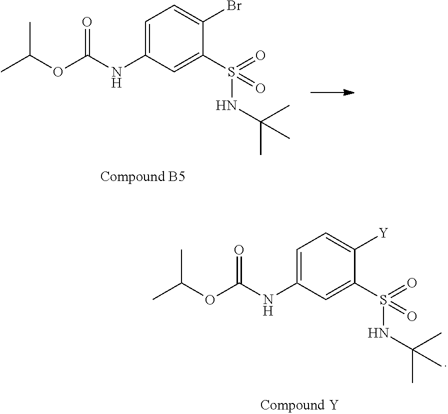

### Step B1

In some embodiments, the present application provides the method of making Compound B2 or a pharmaceutically acceptable salt or solvate thereof. In some embodiments, the present application provides the method of making Compound B2.

In some embodiments, Compound B2 or a pharmaceutically acceptable salt or solvate thereof is prepared by a method disclosed herein. In some embodiments, Compound B2 is prepared by a method disclosed herein.

In some embodiments, Compound B2 is prepared by adding HNO₃ to Compound B1. In some embodiments, the method of the present application comprises Step B1: reacting Compound B1 with HNO₃ to form Compound B2.

In some embodiments, Step B1 comprises adding HNO₃ to Compound B1.

In some embodiments, Step B1 comprises adding an acid to Compound B1. In some embodiments, Step B1 comprises adding sulfuric acid to Compound B1. In some embodiments, Step B1 comprises adding nitric acid to Compound B1. In some embodiments, nitric acid is added dropwise. In some embodiments, nitric acid is added over a time less than about 90 minutes, about 60 minutes, about 30 minutes, about 20 minutes, about 10 minutes, or about 5 minutes.

In some embodiments, Step B1 comprises adding nitric acid to Compound B1 in sulfuric acid. In some embodiments, Step B1 comprises reacting Compound B1 with nitric acid at a temperature below room temperature. In some embodiments, Compound B1 is reacted with nitric acid at temperature between about 0° C. and about 10° C. In some embodiments, Compound B1 is reacted with nitric acid at a temperature of about 0° C., about 5° C., about 10° C., about 15° C., about 20° C., or about 25° C.

In some embodiments, Step B1 may further comprise filtering the solution. In some embodiments, Step B1 may further comprise filtering the solution to collect a solid (e.g., Compound B2). In some embodiments, Step B1 may further comprise washing Compound B2. In some embodiments, Compound B2 is washed with water. In some embodiments, Step B1 may further comprising drying the solid, Compound B2 (e.g., overnight).

In some embodiments, the yield of Compound B2 in Step B1 is at least about 70%, at least about 75%, at least about 80%, at least about 85%, at least about 85%, at least about 90%, or at least about 95%. In some embodiments, the yield of Compound B2 in Step B1 is at least about 70%.

In some embodiments, the purity of Compound B2 in Step B1 is at least about 70%, at least about 75%, at least about 80%, at least about 85%, at least about 85%, at least about 90%, at least about 95%, at least about 98%, or at least about 99%. In some embodiments, the purity of Compound B2 in Step B1 is at least about 95%.

### Step B2

In some embodiments, the present application provides the method of making Compound B3 or a pharmaceutically acceptable salt or solvate thereof. In some embodiments, the present application provides the method of making Compound B3.

In some embodiments, Compound B3 or a pharmaceutically acceptable salt or solvate thereof is prepared by a method disclosed herein. In some embodiments, Compound B3 is prepared by a method disclosed herein.

In some embodiments, Compound B3 is prepared by adding 2-methylpropane-2-amine to Compound B2.

In some embodiments, the method of the present application comprises Step B2: reacting Compound B2 with 2-methylpropane-2-amine, to form Compound B3.

In some embodiments, Step B2 comprises adding 2-methylpropane-2-amine to Compound B2.

In some embodiments, Step B2 comprises adding solvent to Compound B2. In some embodiments, Step B2 comprises adding an aprotic solvent (e.g., 2-MeTHF, ethyl acetate) to Compound B2. In some embodiments, Step B2 comprises adding 2-MeTHF to Compound B2. In some embodiments, Step B2 comprises adding tert-butylamine to Compound B2. In some embodiments, tert-butylamine is added dropwise. In some embodiments, tert-butylamine is added over about 1-3 hours.

In some embodiments, Step B2 comprises adding tert-butylamine to Compound B2 in 2-MeTHF. In some embodiments, Step B2 comprises adding tert-butylamine to Compound B2 in 2-MeTHF at a temperature of about 0-10° C. In some embodiments, Step B2 comprises reacting Compound B2 with tert-butylamine at a temperature of about 20-30° C.

In some embodiments, Step B2 comprises reacting Compound B2 with tert-butylamine for less than about 10 hours. In some embodiments, Compound B2 is reacted with tert-butylamine for less than about 8 hours, less than about 7 hours, less than about 6 hours, less than about 5 hours, less than about 4 hours, less than about 3 hours, less than about 2 hours, or less than about 1 hour. In some embodiments, Compound B2 is reacted with tert-butylamine for about 6 hours.

In some embodiments, Step B2 comprises reacting Compound B2 with tert-butylamine and adjusting the pH to less than about 4, less than about 3, less than about 2 with aqueous HCl solution. In some embodiments, Step B2 comprises reacting Compound B2 with tert-butylamine and adjusting the pH to between 1 and 2 with 1 N aqueous HCl solution.

In some embodiments, Step B2 may further comprise washing the organic layer. In some embodiments, the organic layer is washed with water. In some embodiments, the organic solution is concentrated and isopropyl alcohol is added. In some embodiments, the organic solution of Step B2 is concentrated. In some embodiments, the organic solution of Step B2 is concentrated, cooled and then filtered. In some embodiments, Step B2 may further comprising drying the solid, Compound B3 (e.g., overnight). In some embodiments, the dried Compound B3 is crude. In some embodiments, the crude Compound B3 is recrystallized. In some embodiments, Compound B3 is recrystallized from warm isopropanol.

In some embodiments, the yield of Compound B3 in Step B2 is at least about 70%, at least about 75%, at least about 80%, at least about 85%, at least about 85%, at least about 90%, or at least about 95%. In some embodiments, the yield of Compound B3 in Step B2 is at least about 80%.

In some embodiments, the purity of Compound B3 in Step B2 is at least about 70%, at least about 75%, at least about 80%, at least about 85%, at least about 85%, at least about 90%, at least about 95%, at least about 98%, or at least about 99%. In some embodiments, the purity of Compound B3 in Step B2 is at least about 99%.

### Step B3

In some embodiments, the present application provides the method of making Compound B4 or a pharmaceutically acceptable salt or solvate thereof. In some embodiments, the present application provides the method of making Compound B4.

In some embodiments, Compound B4 or a pharmaceutically acceptable salt or solvate thereof is prepared by a method disclosed herein. In some embodiments, Compound B4 is prepared by a method disclosed herein.

In some embodiments, Compound B4 is prepared by adding hydrogen and a catalyst (e.g., Pt/V/C) to Compound B3.

In some embodiments, Compound B4 is prepared by adding hydrogen and Pt/V/C to Compound B3.

In some embodiments, the method of the present application comprises Step B3: reacting Compound B3, with hydrogen and Pt/C to form Compound B4.

In some embodiments, Step B3 comprises adding hydrogen and Pt/V/C (e.g., 2% Pt, 1% V, 50% water wet) to Compound B3.

In some embodiments, Step B3 comprises adding solvent to Compound B3. In some embodiments, Step B3 comprises adding an aprotic solvent (e.g., 2-MeTHF) to Compound B3. In some embodiments, Step B3 comprises adding 2-MeTHF to Compound B3. In some embodiments, Step B3 comprises adding a catalyst to Compound B3. In some embodiments, Step B3 comprises adding a catalyst to Compound B3 in 2-MeTHF.

In some embodiments, the catalyst of Step B3 comprises platinum on carbon. In some embodiments, the catalyst of Step B3 comprises Pt/C (e.g., 5% Pt/C), Pt/C (e.g., 5% Pt/C) modified with H₃PO₂ and NH₄VO₃, or Fe/NH₄Cl (transfer hydrogenation).

In some embodiments, Step B3 comprises adding a catalyst to Compound B3 and 2-MeTHF in a reaction vessel. In some embodiments, the reaction vessel is purged with hydrogen. In some embodiments, Step B3 comprises placing the reaction vessel under pressure. In some embodiments, the pressure is from about 0.1 MPa to about 1 MPa, from about 0.2 MPa to about 0.4 MPa, or from about 0.3 MPa to about 0.4 MPa.

In some embodiments, Step B3 comprises reacting Compound B3, catalyst, and hydrogen under pressure at a temperature below about 70° C.

In some embodiments, Compound B3, catalyst, and hydrogen are reacted at a temperature of about 40° C., about 45° C., about 50° C., about 55° C., about 60° C., about 65° C., or about 70° C.

In some embodiments, Step B3 comprises reacting Compound B3, catalyst, and hydrogen for less than about 24 hours, less than about 20 hours, less than about 16 hours, less than about 14 hours, or less than about 10 hours. In some embodiments, Step B3 comprises reacting Compound B3, catalyst, and hydrogen for about 14 hours.

In some embodiments, Step B3 comprises reacting Compound B3, catalyst, and hydrogen at a temperature of about 45-55° C. for about 14 hours at a pressure from about 0.3 MPa to about 0.4 MPa.

In some embodiments, Step B3 may further comprise filtering the reaction. In some embodiments, the reaction is filtered through diatomite filter.

In some embodiments, the filter cake (i.e., Compound B4) is washed with solvent. In some embodiments, the filter cake (i.e., Compound B4) is washed with aprotic solvent. In some embodiments, Compound B4 is washed with 2-MeTHF. In some embodiments, Step B3 may further comprising drying the solid, Compound B4 (e.g., overnight).

In some embodiments, the yield of Compound B4 in Step B3 is at least about 70%, at least about 75%, at least about 80%, at least about 85%, at least about 85%, at least about 90%, or at least about 95%. In some embodiments, the yield of Compound B4 in Step B3 is at least about 90%.

In some embodiments, the purity of Compound B4 in Step B3 is at least about 70%, at least about 75%, at least about 80%, at least about 85%, at least about 85%, at least about 90%, at least about 95%, at least about 98%, or at least about 99%. In some embodiments, the purity of Compound B4 in Step B3 is at least about 99%.

### Step B4

In some embodiments, the present application provides the method of making Compound B5 or a pharmaceutically acceptable salt or solvate thereof. In some embodiments, the present application provides the method of making Compound B5.

In some embodiments, Compound B5 or a pharmaceutically acceptable salt or solvate thereof is prepared by a method disclosed herein. In some embodiments, Compound B5 is prepared by a method disclosed herein.

In some embodiments, Compound B5 is prepared by adding isopropyl chloroformate to Compound B4.

In some embodiments, the method of the present application comprises Step B4: reacting Compound B4 with isopropyl chloroformate to form Compound B5.

In some embodiments, Step B4 comprises adding isopropyl chloroformate to Compound B4.

In some embodiments, Step B4 comprises adding solvent to Compound B4. In some embodiments, Step B4 comprises adding an aprotic solvent (e.g., 2-MeTHF) to Compound B4. In some embodiments, Step B4 comprises adding 2-MeTHF to Compound B4. In some embodiments, Step B4 comprises adding di-isopropyl ethylamine (DIPEA) to Compound B4 and 2-MeTHF.

In some embodiments, Step B4 comprises adding the isopropyl chloroformate to the reaction mixture of Compound B4. In some embodiments, the isopropyl chloroformate mixture is added dropwise.

In some embodiments, Step B4 comprises stirring the reaction mixture at a temperature above room temperature. In some embodiments, Step B4 comprises stirring the reaction mixture at a temperature of about 65-75° C. In some embodiments, Step B4 comprises stirring the reaction mixture at a temperature of about 65-75° C. for about 10 hours, about 8 hours, about 6 hours, about 4 hours, or about 2 hours.

In some embodiments, Step B4 may further comprise washing the reaction mixture. In some embodiments, Step B4 may further comprise washing the reaction mixture with acid. In some embodiments, Step B4 may further comprise washing the reaction with 1N hydrogen chloride to pH of about 1-2. In some embodiments, Step B4 may further comprise washing the organic layer.

In some embodiments, the organic layer is washed with water. In some embodiments, the organic solution is concentrated and heptane is added. In some embodiments, Step B4 may further comprise filtering the precipitate. In some embodiments, Step B4 may further comprise drying the solid, Compound B5 (e.g., overnight).

In some embodiments, the yield of Compound B5 in Step B4 is at least about 70%, at least about 75%, at least about 80%, at least about 85%, at least about 85%, at least about 90%, or at least about 95%. In some embodiments, the yield of Compound B5 in Step B4 is at least 90%.

In some embodiments, the purity of Compound B5 in Step B4 is at least about 70%, at least about 75%, at least about 80%, at least about 85%, at least about 85%, at least about 90%, at least about 95%, at least about 98%, or at least about 99%. In some embodiments, the purity of Compound B5 in Step B4 is at least about 99%.

### Step B5

In some embodiments, the present application provides the method of making Compound Y or a pharmaceutically acceptable salt or solvate thereof. In some embodiments, the present application provides the method of making Compound Y.

In some embodiments, Compound Y or a pharmaceutically acceptable salt or solvate thereof is prepared by a method disclosed herein. In some embodiments, Compound Y is prepared by a method disclosed herein.

In some embodiments, Compound Y is prepared by adding B₂(OH)₄ to Compound B5.

In some embodiments, the method of the present application comprises Step B5: reacting Compound B5 with B₂(OH)₄ to form Compound Y.

In some embodiments, Step B5 comprises adding B₂(OH)₄ to Compound B5.

In some embodiments, Step B5 comprises adding a catalyst to Compound B5. In some embodiments, Step B5 comprises adding B₂(OH)₄ (i.e., methanolic solution of B₂(OH)₄), potassium acetate, and bis(triphenylphosphine)palladium (II) dichloride (catalyst 2) to Compound B5. In some embodiments, Step B5 comprises adding solvent to Compound B5. In some embodiments, Step B5 comprises adding an aprotic solvent (e.g., 2-MeTHF, methanol, ethanol, iso-propanol, dioxane, dimethylacetamide, and toluene, or a mixture thereof) to Compound B5. In some embodiments, Step B5 comprises adding 2-MeTHF to Compound B5. In some embodiments, Step B5 comprises adding methanol to Compound B5. In some embodiments, Step B5 comprises adding 2-MeTHF and methanol to Compound B5. In some embodiments, Step B5 comprises adding potassium acetate, 2-MeTHF and methanol to Compound B5.

In some embodiments, Step B5 comprises stirring the reaction mixture at a temperature below about 100° C. In some embodiments, Compound B5, potassium acetate, 2-MeTHF, methanol, the methanolic solution of B₂(OH)₄, and catalyst 2 are reacted at a temperature of about 50° C., about 55° C., about 60° C., about 65° C., about 70° C., about 75° C., about 80° C., about 85° C., about 90° C., about 95° C., or about 100° C.

In some embodiments, Step B5 comprises stirring the reaction mixture for less than about 40 hours. In some embodiments, Compound B5 is reacted with catalyst 2 for less than about 36 hours, less than about 30 hours, less than about 24 hours, or less than about 20 hours. In some embodiments, Compound B5 is reacted with catalyst 2 for about 20 hours, about 19 hours, about 18 hours, about 17 hours, about 16 hours, or about 15 hours. In some embodiments, Compound B5 is reacted with catalyst 2 for about 16 hours. In some embodiments, Compound B5 is reacted with methanolic solution of B₂(OH)₄ in the presence of catalyst 2 for about 16 hours in 2-MeTHF, KOAc, and methanol.

In some embodiments, the solution of Step B5 is filtered. In some embodiments, the solution is filtered through a CUNO™ filter. In some embodiments, the filter cake of Step B5 is washed. In some embodiments, the filter cake of Step B5 is washed with EtOAc.

In some embodiments, the organic solution of Step B5 is concentrated and washed. In some embodiments, the resultant precipitate is collected via filtration. In some embodiments, Step A5 may further comprising drying the solid, Compound Y (e.g., overnight).

In some embodiments, the yield of Compound Y in Step B5 is at least about 70%, at least about 75%, at least about 80%, at least about 85%, at least about 85%, at least about 90%, or at least about 95%. In some embodiments, the yield of Compound Y in Step B5 is at least about 85%.

In some embodiments, the purity of Compound Y in Step B5 is at least about 70%, at least about 75%, at least about 80%, at least about 85%, at least about 85%, at least about 90%, at least about 95%, at least about 98%, or at least about 99%. In some embodiments, the purity of Compound Y in Step B5 is at least about 99%.

Embodiment 20: In some aspects, in Embodiment 19, Step B5 is as follows:

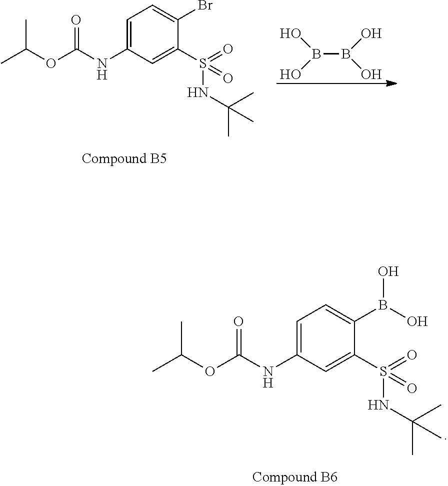 [SMILES: CC(C)OC(=O)Nc1ccc(Br)c(S(=O)(=O)NC(C)(C)C)c1 ; OB(O)B(O)O ; CC(C)OC(=O)Nc1ccc(B(O)O)c(S(=O)(=O)NC(C)(C)C)c1]

In some embodiments, the present application provides the method of making Compound B6 or a pharmaceutically acceptable salt or solvate thereof. In some embodiments, the present application provides the method of making Compound B6.

In some embodiments, Compound B6 or a pharmaceutically acceptable salt or solvate thereof is prepared by a method disclosed herein. In some embodiments, Compound B6 is prepared by a method disclosed herein.

In some embodiments, Compound B6 is prepared by adding B₂(OH)₄ to Compound B5.

In some embodiments, the method of the present application comprises Step B5: reacting Compound B5 with B₂(OH)₄ to form Compound B6.

In some embodiments, Step A5 may further comprising drying the solid, Compound B6 (e.g., overnight).

In some embodiments, the yield of Compound B6 in Step B5 is at least about 65%, at least about 70%, at least about 75%, at least about 80%, at least about 85%, at least about 85%, at least about 90%, or at least about 95%. In some embodiments, the yield of Compound B6 in Step B5 is at least about 85%.

In some embodiments, the purity of Compound B6 in Step B5 is at least about 70%, at least about 75%, at least about 80%, at least about 85%, at least about 85%, at least about 90%, at least about 95%, at least about 98%, or at least about 99%. In some embodiments, the purity of Compound B6 in Step B5 is at least about 99%.

Embodiment 21: In some aspects, in Embodiment 19, Step B5 is as follows:

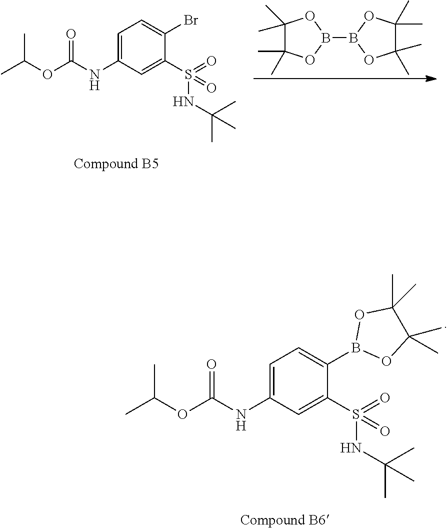

### Step B5 (Alternative Route)

In some embodiments, Step B5 comprises reacting Compound B5 with B₂Pin₂ in the presence of palladium acetate and XPhos in DMF affords the boronate ester which is expected to react with Compound X in a comparable manner to Compound B6′.

Embodiment 22: In some aspects, in any one of Embodiments 1-21, Step (a) or Step (b) is carried out in the presence of a palladium catalyst.

Embodiment 23: In some aspects, in Embodiment 22, the palladium catalyst is selected from:

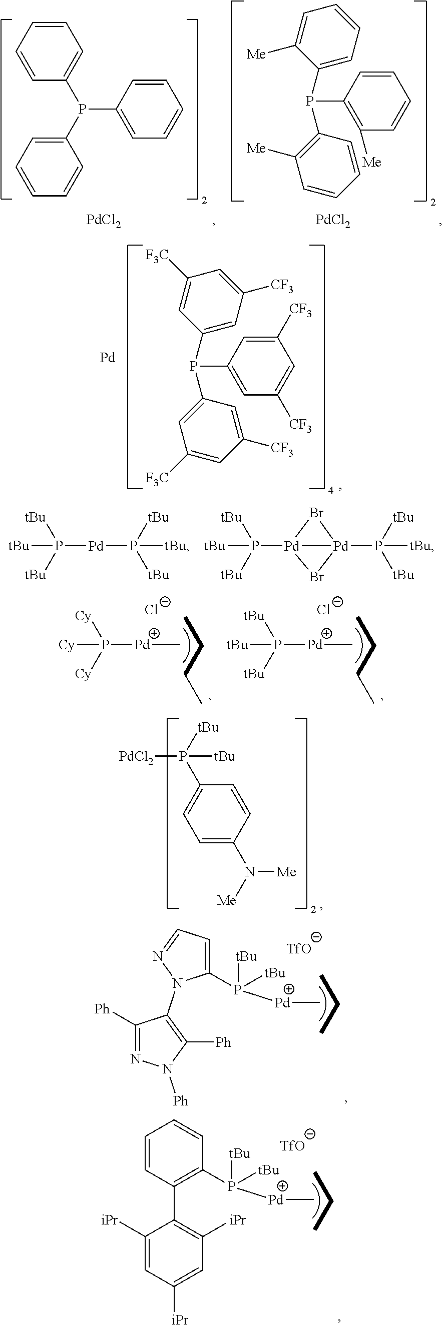

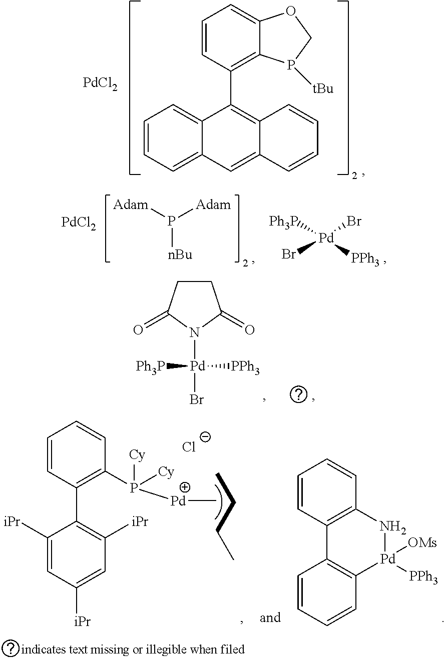

In some embodiments, the method of preparing Compound A6 of the present application comprises one or more steps shown in Scheme I below:

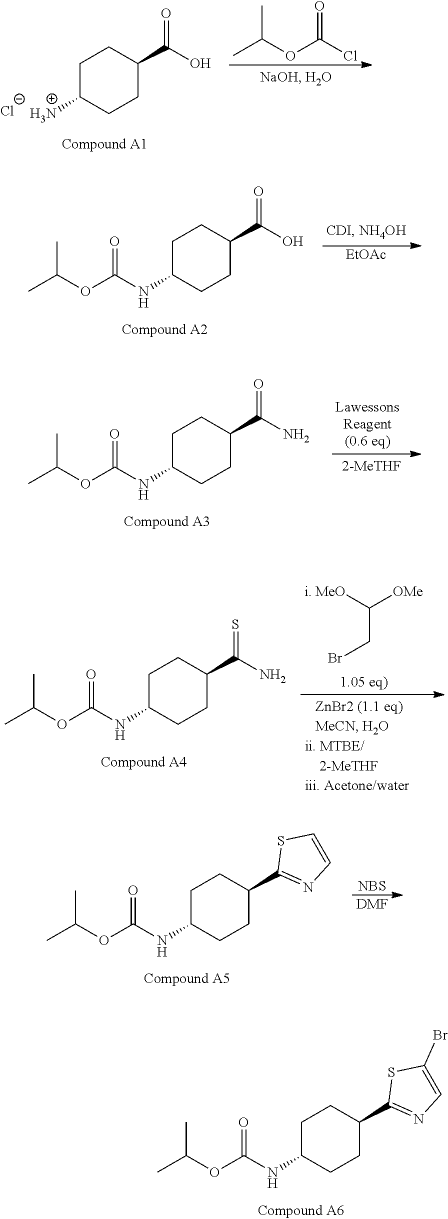

In some embodiments, the method of preparing Compound B6 of the present application comprises one or more steps shown in Scheme II below:

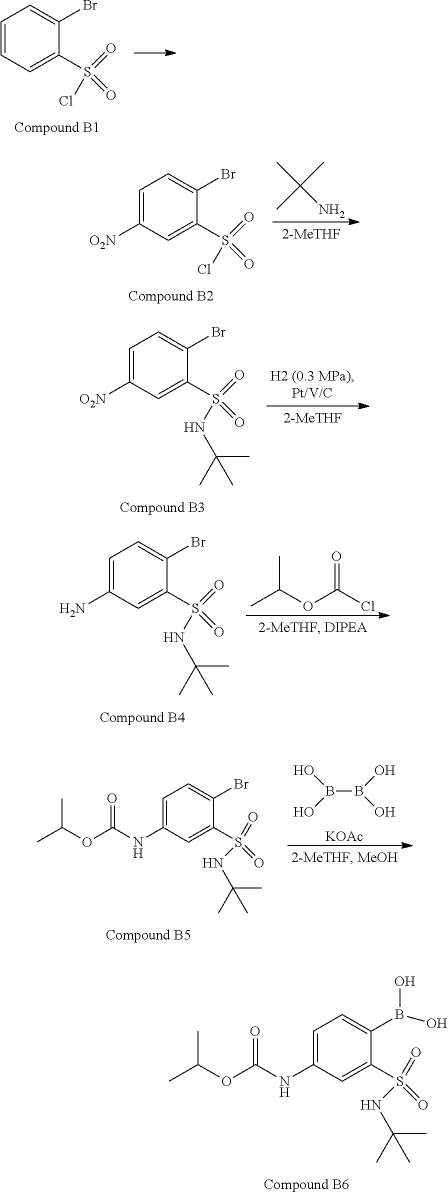

In some embodiments, the method of preparing Compound A of the present application comprises one or more steps shown in Scheme III below:

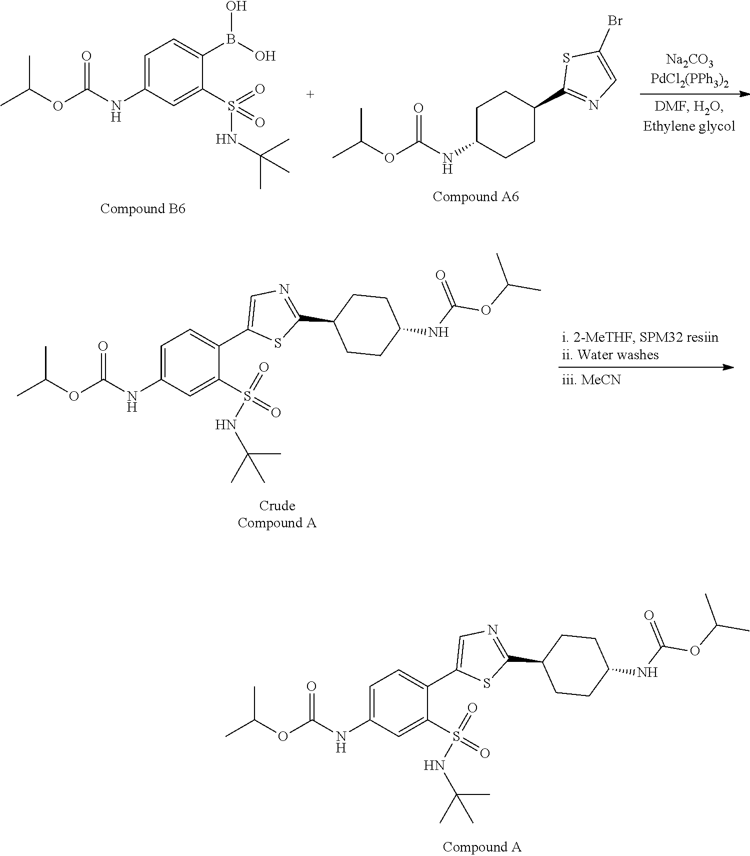

In some embodiments, the methods of the present application produce Compound A or a pharmaceutically acceptable salt or solvate thereof at a yield of at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the methods of the present application produce Compound A or a pharmaceutically acceptable salt or solvate thereof at a yield of at least about 60%, at least about 65%, at least about 70%, at least about 75%, at least about 80%, at least about 85%, at least about 90%, or at least about 95%.

In some embodiments, the methods of the present application produce substantially pure Compound A or a pharmaceutically acceptable salt or solvate thereof. The term “purity” as used herein refers to the amount of Compound A or a pharmaceutically acceptable salt or solvate thereof based on analytic methods commonly used in the art (e.g., high-performance liquid chromatography (HPLC), nuclear magnetic resonance spectroscopy (NMR), mass spectroscopy, and X-ray powder diffraction (XRPD)). Purity is based on the “organic” purity of the compound, and does not include a measure of any amount of water, solvent, etc. In some embodiments, the purity of Compound A or a pharmaceutically acceptable salt or solvate thereof is compared to the purity of a reference standard, e.g. a known sample of Compound A by comparing the area under the peak in HPLC. In some embodiments, Compound A or a pharmaceutically acceptable salt or solvate thereof prepared according to the methods of the present application has a purity of greater than about 96%. In some embodiments, Compound A or a pharmaceutically acceptable salt or solvate thereof prepared according to the methods of the present application has a purity of greater than about 98%. For example, the purity of Compound A or a pharmaceutically acceptable salt or solvate thereof prepared according to the methods of the present application is 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%. For example, the purity of Compound A or a pharmaceutically acceptable salt or solvate thereof prepared according to the methods of the present application is 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%. For example, the purity of Compound A or a pharmaceutically acceptable salt or solvate thereof prepared according to the methods of the present application is 98.0%, 98.5%, 99.0%, 99.5%, 99.6%, 99.7%, 99.8%, or 99.9%.

In some embodiments, the methods of the present application produce highly pure Compound A or a pharmaceutically acceptable salt or solvate thereof on a large scale (e.g., commercial scale). The term “commercial scale” refers to yield of a single batch of at least about 100 g. In some embodiments, the methods of the present application produce highly pure Compound A or a pharmaceutically acceptable salt or solvate thereof in a large amount of at least about 25 g, at least about 50 g, at least about 75 g, at least about 100 g, at least about 150 g, at least about 200 g, at least about 250 g, at least about 300 g, at least about 350 g, at least about 400 g, at least about 450 g, at least about 500 g, at least about 1 kg, at least about 2 kg, at least about 5 kg, at least about 10 kg, at least about 15 kg, at least about 20 kg, at least about 25 kg, or at least about 30 kg.

Any numerals used in this disclosure with or without about/approximately are meant to cover any normal fluctuations appreciated by one of ordinary skill in the relevant art. In some embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

### Pharmaceutical Compositions

The present application also provides pharmaceutical compositions comprising Compound A or a pharmaceutically acceptable salt or solvate thereof prepared by the methods of the present application, in combination with at least one pharmaceutically acceptable excipient or carrier.

A “pharmaceutical composition” is a formulation containing the compound of the present application in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or a pharmaceutically acceptable salt or solvate thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In some embodiments, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” and “pharmaceutically acceptable diluent” refer to a substance that aids the formulation and/or administration of an active agent to and/or absorption by a subject and can be included in the compositions of the present application without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The term “carrier”, as used in this application, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

The term “carrier”, as used in this application, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

### Definitions

As used herein, the term “substantially pure” with reference to a compound indicates that the compound includes less than 10%, less than 5%, less than 3%, less than 1%, less than 0.5%, less than 0.2%, or less than 0.1% by weight of impurities.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” or “therapeutically effective amount” when used in connection with a compound or pharmaceutical composition is an amount effective for treating or preventing a disease in a subject as described herein.

The term “treating” with regard to a subject, refers to improving at least one symptom of the subject's disorder. Treating includes curing, improving, or at least partially ameliorating the disorder.

The term “disorder” is used in this application to mean, and is used interchangeably with, the terms disease, condition, or illness, unless otherwise indicated.

The present application provides use of Compound A or a pharmaceutically acceptable salt or solvate thereof that functions as modulator of MCT activity.

The present application provides a method of treating or preventing a disease or disorder associated with the abnormal expression or activity of monocarboxylate transporters, or dependency on the expression or activity of at least one MCT.

As used herein, the term diseases or disorders associated with the abnormal expression or activity of monocarboxylate transporters, or dependency on the expression or activity of at least one MCT means any disease or other deleterious condition in which MCT modulation is known to play a role. Accordingly, another embodiment of the present application relates to treating or lessening the severity of one or more diseases in which MCT modulation is known to play a role.

As used herein the term “organic solvent” refers to an organic molecule capable of at least partially dissolving another substance (i.e., the solute). Examples of organic solvents that may be used for the present invention include, but are not limited to: hydrocarbon solvents (e.g., n-pentane, n-hexane, n-heptane, n-octane, paraffin, cyclohexane, methylcyclohexane, decahydronaphthalene, mineral oil, crude oils, etc.) which also includes aromatic hydrocarbon solvents (e.g., benzene, toluene, o-xylene, m-xylene, and p-xylene), halogenated hydrocarbon solvents (e.g., carbon tetrachloride, 1,2-dichloroethane, dichloromethane, chloroform, etc.), ester solvents (e.g., ethyl formate, methyl acetate, ethyl acetate, isopropyl acetate, butyl acetate, isobutyl acetate, ethyl malonate, etc.), ketone solvents (e.g., acetone, methyl ethyl ketone or 2-butanone, methyl isobutyl ketone, cyclohexanone, cyclopentanone, 3-pentanone, etc.), ether solvents (e.g., diethyl ether, dipropyl ether, diphenyl ether, isopropyl ether, tert-butyl methyl ether, tetrahydrofuran, 1,4-dioxane, etc.), amine solvents (e.g., propyl amine, diethylamine, triethylamine, aniline, pyridine), alcohol solvents (e.g., methanol, ethanol, isopropanol, 1-propanol, 2-methyl-1-propanol, 1-butanol, 2-butanol, 1-pentanol, 3-methyl-1-butanol, tert-butanol, 1-octanol, benzyl alcohol, phenol, trifluoroethanol, glycerol, ethylene glycol, propylene glycol, m-cresol, etc.), acid solvents (e.g., acetic acid, hexanoic acid, etc.), carbon disulfide, nitrobenzene, N,N-dimethylformamide, N,N,-dimethylacetamide, dimethyl sulfoxide, N-methyl-2-pyrrolidone, acetonitrile, silicone solvents (e.g., silicone oils, polysiloxanes, cyclosilicones). In some embodiments, the organic solvent may be formed by the combination of two or more organic solvents.

As used herein, unless otherwise noted, the term “aprotic solvent” refers to any solvent that does not yield a proton.

The term “protic solvent” refers to any molecular solvent which contains dissociable H⁺.

All percentages and ratios used herein, unless otherwise indicated, are by weight. Other features and advantages of the present application are apparent from the different examples. The provided examples illustrate different components and methodology useful in practicing the present application. The examples do not limit the claimed disclosure. Based on the present application the skilled artisan can identify and employ other components and methodology useful for practicing the present application.

### EXAMPLES

### Example 1. Synthesis of Compound A2

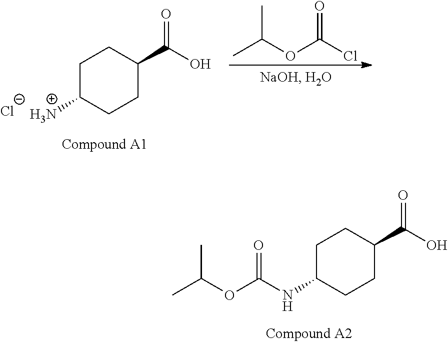

Water (1.08 L) and aqueous sodium hydroxide solution (396 g, 30 wt %, 4.1 eq.) were charged into a suitable reactor and the mixture was stirred at 0-10° C. Compound A1 (131 g, 1.0 eq.) was charged at 0-10° C., followed by isopropyl chloroformate (128 g, 1.43 eq.) while maintaining the reaction mixture at 0-25° C.

Upon reaction completion (˜ 4-6 hours), the mixture was cooled to 0-10° C., quenched with 6M aq. HCl solution (244 g) and stirred at 0-10° C. for 6 hours. The precipitated solids were collected by filtration and the filter cake was washed with water (760 mL). The product was dried at 45-65° C. for 5 days to give 154 g of Compound A2 with a purity of 98.3% and in 90% corrected yield.

### Example 2. Synthesis of Compound A2 Alternative Procedure Using Phase Transfer Conditions

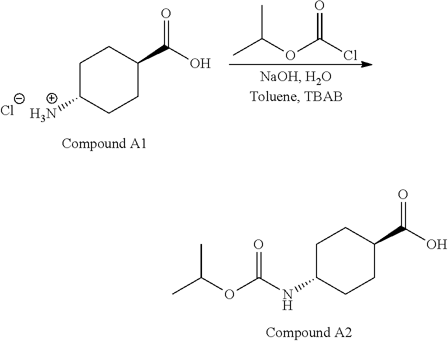

Water (500 mL) and sodium hydroxide solution (21.2 g, 0.85 eq) were charged into a suitable reactor and the mixture was stirred at 0-20° C. Compound A1 (25 g, 1.0 eq.) was added followed by tetrabutylammonium bromide (TBAB) (4.5 g 0.18 eq) at the same temperature. A solution of isopropyl chloroformate (23.9 g, 0.98 eq.) in toluene (168 g) was added while maintaining the reaction mixture at 0-20° C. The mixture was then stirred at 15-25° C. for 24 hours and checked for reaction completion.

Upon reaction completion ethyl acetate was added, the mixture was first stirred, then allowed to stand and the resulting layers separated. The aqueous layer was acidified with 6M aq. HCl solution (75 g) and stirred at 0-10° C. for 6 hours. The precipitated solids were collected by filtration and the filter cake was washed with water (3×150 mL). The product was dried at 45-55° C. for 24 hours to give Compound A2 in 85% yield.

### Example 3. Synthesis of Compound A3

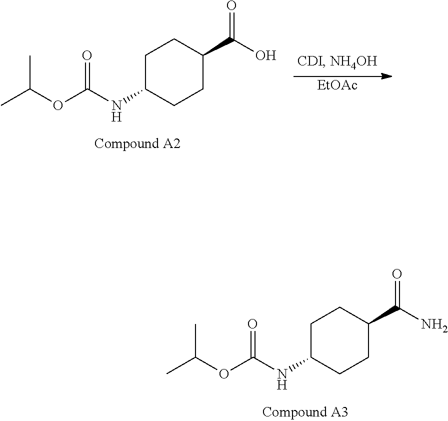 [SMILES: "EtOAc" ; CC(C)OC(=O)N[C@H]1CC[C@H](C(=O)O)CC1 ; CC(C)OC(=O)N[C@H]1CC[C@H](C(N)=O)CC1]

Compound A2 (140 g, 1.0 eq.) and EtOAc (2.66 L) were charged to a suitable reactor and the mixture was stirred at 15-25° C., followed by addition of CDI (151 g, 1.5 eq.). The mixture was stirred at 15-25° C. for 3 hours to ensure conversion to the activated intermediate.

Ammonium hydroxide (25 wt %, 428 g, 5.0 eq.) was charged at 15-25° C., and stirring continued at the same temperature for 5 hours, after which time>99% conversion of Compound A2 to the desired Compound A3 had been achieved.

The solids were collected by filtration and the filter cake was rinsed with water (635 mL) to provide crude Compound A3 (475 g). Crude Compound A3 was charged back to the reactor, followed by the addition of MeCN (600 g) and process water (700 mL). The slurry was stirred at 15-25° C. for 6 hours prior to filtration. The filter cake was rinsed with MeCN (497 g) to provide wet Compound A3 with a purity of 98.4%. This was dried at 60-70° C. for 48 h to give 133 g of Compound A3 in ˜99% purity, 97% assay and 91% corrected yield.

### Example 4. Synthesis of Compound A3 Using Iso-Propylchloroformate

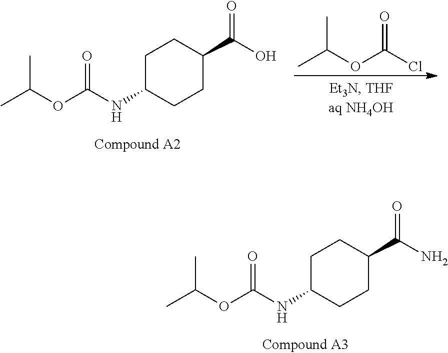

Compound A2 (53.5 g, 1.0 eq.) and triethylamine (28.7 g, 1.3 eq) and THF (550 mL) were added to a suitable vessel and the mixture was stirred at −20° C., followed by addition of isopropylchloroformate (32.1 g, 1.2 eq). The mixture was stirred at −15° C. for 30 minutes and then 30 wt % ammonium hydroxide (764 g, 25 eq) added at the same temperature and then warmed to 15-25° C., and stirring continued at the same temperature for 2-3 days.

The reaction mixture was concentrated under vacuum to remove the THF and the solids isolated by filtration and washed with water (250 mL) and 1:9 v: IPA: Heptane (500 mL). The solids were dried at 45-55° C. under vacuum for 18 hours to afford Compound A3 in 75% yield.

### Example 5. Synthesis of Compound A4

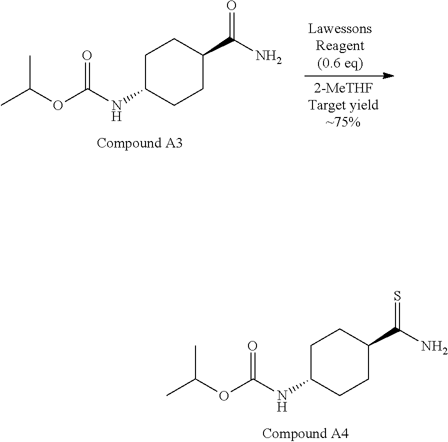 [SMILES: CC(C)OC(=O)N[C@H]1CC[C@H](C(N)=O)CC1 ; CC(C)OC(=O)N[C@H]1CC[C@H](C(N)=S)CC1]

Compound A3 (100 g, 1.0 eq.) and 2-MeTHF (1290 g) were charged to a suitable reactor under a nitrogen atmosphere and the mixture was stirred at 0-10° C. Lawesson's reagent (105 g, 0.61 eq.) was charged at 0-10° C. The reaction was stirred at 20-30° C. for 24 hours when a sample showed the reaction had reached >99% conversion.

Upon reaction completion, the mixture was filtered to remove insoluble materials and rinsed with 2-MeTHF (172 g). 10% Na₂CO₃ aqueous solution (600 g) was added at 15-25° C., the mixture stirred and the layers allowed to separate. After removal of the aqueous layer, the 2-MeTHF solution was washed with water twice (500 mL then 300 mL).

The final 2-MeTHF solution was then distilled under vacuum to 3-5 vols keeping the internal temperature below 50° C. Further 2-MeTHF (430 g) was added and then further concentrated to 4-5 vols. The mixture was heated to 45-55° C. to obtain a clear solution and then cooled to 35-45° C. and seeded with 0.5 wt % of Compound A4 and stirred for a further hour at the same temperature. Heptane (680 g) was charged at this temperature. The slurry was agitated at 45-55° C. for 4 hours, cooled to 15-25° C. over 4 hours and agitated at 15-25° C. for 14 hours. The solids were collected by filtration and the filter cake was rinsed with heptane (172 g) to give wet Compound A4. The product was dried at 45-55° C. for 19 hours to give 95 g of Compound A4 with 92.7% purity, 84.4% assay and in 75% (assay adjusted) yield.

This reaction can also be run in other solvents including but not limited to dichloromethane, dichloroethane and dimethoxy ethane as well as mixtures of the above.

### Example 6. Synthesis of Compound A5 Using Zinc Bromide

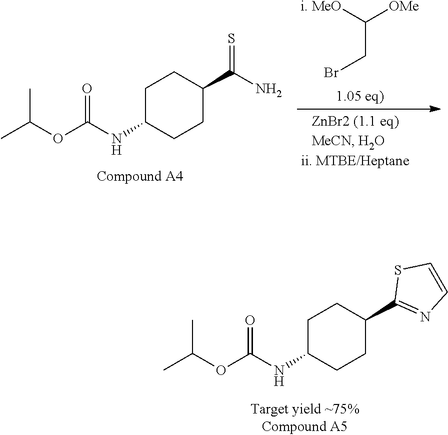

Compound A4 (117.6 g, 100.0 g corrected for assay, 1 eq), MeCN (790.0 g) and purified water (7.36 g) were charged to an appropriate vessel under a nitrogen atmosphere at 15-25° C. and stirring started. 2-bromo-1,1-dimethoxy-ethane (80.9 g, 1.17 eq), followed by ZnBr₂ (101.39 g, 1.1 eq) were added at the same temperature. The reaction mixture was heated to 70-80° C. for 5-9 hours after which time HPLC analysis indicated >99% conversion.

The reaction mixture was distilled under vacuum to 3-4 vols keeping the internal temperature below and then cooled to 15-25° C. 18% Na₂CO₃ aqueous solution (132.0 g) was added slowly at 15-25° C. followed by MTBE (1554.0 g). A further charge of 18% Na₂CO₃ aqueous solution (400.0 g) was added and the mixture stirred for 1-2 hours. The solids were removed by filtration and washed with MTBE (185.0 g).

The filtrate was returned to the vessel, stirred and then the two layers allowed to separate, and the lower layer removed. The upper organic layer is washed with water (500 g) and the layers separated.

The organic layer is decolorized with CUNO™ for 6-24 h and the CUNO™ washed with further MTBE (370.0 g).

The MTBE solution is concentrated under vacuum to 4-5 volumes and then solvent swapped to heptane using three portions of heptane (340 g) distilling to a final heptane volume of volumes (1 L). The slurry is heated to 40-50° C. and 2-MeTHF (86 g) added. After holding at the same temperature for 2 hours the slurry is cooled to 15-25° C. and held for 6-8 h. The solids are filtered and washed with n-heptane (136 g) to give crude Compound A5.

The crude solids are added to a suitable reactor and acetone (316 g) added and the temperature adjusted to 15-25° C. Water (1.60 L) was added over 4 h at the same temperature and the slurry stirred for a further 4 hours. The solids were filtered and washed with water (200 g,) and then dried at 45-55° C. for 18-20 h to give 71.4 g of Compound A5 in 65% yield.

### Example 7. Synthesis of Compound A5 Using Ytterbium Triflate

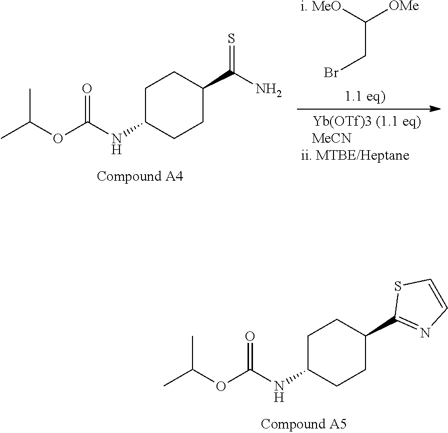

Compound A4 (74.5 g, 1 eq), MeCN (1.11 kg) and Ytterbium triflate (217 g, 1.1 eq) were charged to an appropriate vessel under a nitrogen atmosphere at room temperature and stirring started. 2-bromo-1,1-dimethoxy-ethane (54.2 g, 1.05 eq), was added and the mixture heated to 75-85° C. for 5-9 hours after which time HPLC analysis indicated >99% conversion.

The reaction mixture was distilled under vacuum to 2-3 vols keeping the internal temperature below 45° C. and then cooled to 15-25° C. 7% NaHCO₃ aqueous solution (131.0 g) was added at 15-25° C. which led to precipitation of solids. After 3 hours the solids were collected by filtration and washed with water (131 mL) and then suspended in MTBE (1.15 kg) stirred further and any remaining solids removed by filtration with further washes with MTBE (263 g).

The combined MTBE layers were decolorized with CUNO™ for 6-24 h. The MTBE solution is washed with water (3-10×400 g) and the layers separated each time.

The MTBE solution is concentrated under vacuum to 2-3 volumes and then heptane (501 g) was added at 15-25° C. The slurry was stirred at the same temperature for 5 hours. The solids are filtered and washed with n-heptane (577 g) to give crude Compound A5.

These crude solids are added to a suitable reactor and acetone (235 g) added and the temperature adjusted to 15-25° C. Water (1.30 L) was added over 4 h at the same temperature and the slurry stirred for a further 4 hours. The solids were filtered and washed with water (200 g,) and then dried at 45-55° C. for 18-20 h to give 46.4 g of Compound A5 in 57% yield.

### Example 8. Synthesis of Compound A5 Using Different Reactions

A summary of selected results from screening reactions for this transformation are shown in Table 2. Unless stated all reactions use MeCN as solvent, 2-bromo-1,1-dimethoxyethane as reagent with a reaction temperature of 80° C.

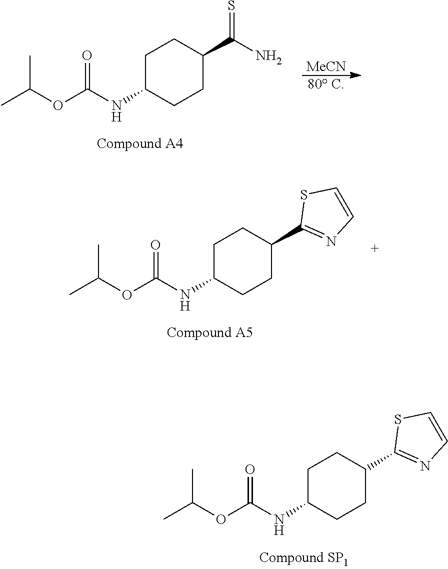

Compound A4, MeCN and the required acid/Lewis acid were charged to an appropriate vessel under a nitrogen atmosphere at room temperature and stirring started. 2-bromo-1,1-dimethoxy-ethane or an alternate reagent was added and the mixture heated to 75-85° C. for 5-9 hours. Reaction conversion and selectivity was measured using HPLC and/or NMR analysis.

TABLE 2

| | | Combined | |
|---|---|---|---|
| | | Compound | Compound |
| Acid/Lewis | | A5 & Compound | A5:Compound |
| Acid | Conditions | SP₁Formed | SP₁ |
| Yb(OTf)₃•H₂O | | 79 | 92:8 |
| Y(OTf)₃ | | 77 | 88:12 |
| Sc(OTf)₃ | | 49 | 92:8 |
| Zn(OTf)₂ | | 80 | 65:35 |
| FeCl₂ | | <5 | Not measured |
| FeCl₂ | Reagent = | 80 | 80:20 |
| | Bromoacetaldehyde | | |
| CoCl₂ | | 61 | 79:21 |
| CeCl₃ | | 80 | 50:50 |
| YbCl₃•6H₂O | | | |
| TiCl₄ | | 36 | 65:35 |
| BF₃•OEt₂ | | 13 | 65:35 |
| p-TSA | | 8 | 65:35 |
| p-TSA | Reagent = | 80 | 50:50 |
| | Bromoacetaldehyde | | |
| Acetic acid | Reagent = | 45 | 75:25 |
| | Bromoacetaldehyde | | |
| CSA | Reagent = | 87 | 75:25 |
| | Bromoacetaldehyde | | |
| CSA | | 29 | 58:42 |
| Aq HCl | | 27 | 57:43 |
| HCl | | <5% | Not measured |
| (anhydrous) | | | |

### Example 9. Alternate Synthetic Approaches to Compound A5

Hydrogenation of CYT-2433 will also afford Compound A5.

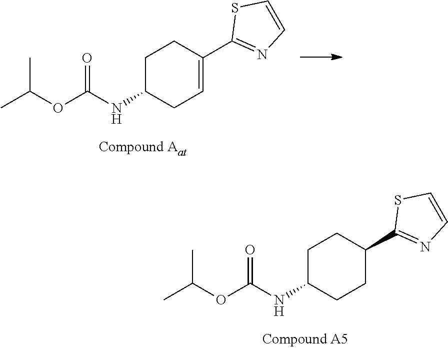 [SMILES: CC(C)OC(=O)N[C@H]1CC=C(c2nccs2)CC1 ; CC(C)OC(=O)N[C@H]1CC[C@H](c2nccs2)CC1]

Conditions for this transformation include but are not limited to Pd/C, (PPh₃)₃RhCl (Wilkinson's Catalyst), Pd(OH)₂, PtO₂, or [C₈H₁₂IrP(C₆H₁₁)₃C₅H₅N]PF₆ (Crabtree's catalyst).

### Example 10. Synthesis of Compound A6

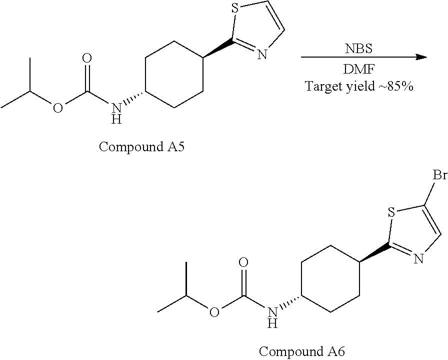

Compound A5 (44.3 g, 1.0 eq.) and DMF (253 g) were charged to a suitable reactor and stirred for 1 hour under a nitrogen atmosphere at 15-25° C. to give a clear solution. A solution of NBS (32.4-35.3 g, 1.1-1.2 eq.) in DMF (125 g) was added at 15-25° C. over 5 hours. After complete addition, the mixture was stirred for a further 16 hours at the same temperature after which time HPLC analysis showed <9% remaining Compound A5. The mixture was cooled to 5-15° C. and water (885 mL) was charged within 6 hours while maintaining a temperature≤ 35° C., followed by adjusting the temperature to 15-25° C. The mixture was stirred at 15-25° C. for 5.5 h. The solid was collected by filtration and the filter cake was washed with water twice (445 mL and 280 mL) and Heptane (256 g). The wet cake was dried at 30-40° C. for 23 hours give crude Compound A6.

The crude Compound A6 was suspended in MTBE (205 g) and heated to 50-60° C. to give a clear solution. Then the mixture was cooled to 40-50° C., and optional Compound A6 seed crystals (0.21 g) were charged, followed by holding the temperature of 40-50° C. for 30 minutes prior to cooling to −5-5° C. over 3 hours. The mixture was agitated at −5-5° C. for 1 hour, and Heptane (189 g) was charged over 4 hours. After agitation at −5-5° C. for 2 hours, the mixture was filtered and the filter cake was rinsed with Heptane (29 g). After drying at 30-40° C. for 24 hours 46.42 g of Compound A6 was isolated with a purity of 99.3%, and in 81% yield.

Alternate reagents for this transformation include but are not limited to, bromine, hydrogen bromide, carbon tetrabromide.

### Example 11. Alternative Syntheses to Compound X

Reaction of Compound A5 with N-iodosucinimde or N-chlorosuccinimide should afford the corresponding iodo- and chlor-derivatives which are also expected to react with boronic acids.

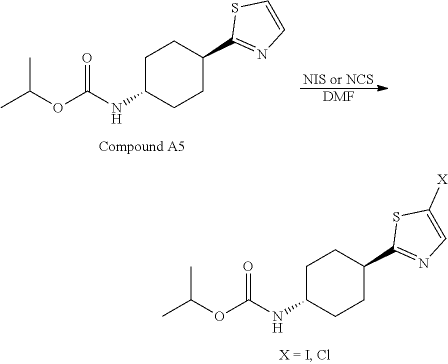

### Example 12. Synthesis of Compound B2

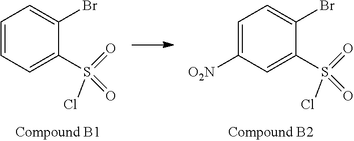 [SMILES: O=S(=O)(Cl)c1ccccc1Br ; O=[N+]([O-])c1ccc(Br)c(S(=O)(=O)Cl)c1]

A suspension of Compound B1 (120 g, 1 eq) in H₂SO₄ (600 mL) was cooled to 0-10° C. with stirring. Then HNO₃ (46.7 g, 95% purity) was added dropwise at 0-10° C. over 1 hour. After addition the reaction mixture was stirred at 0-10° C. for a further 1 hour. The reaction mixture was poured into ice water (1.5 L) slowly at 0-10° C. The resulting mixture was filtered and the filter cake was washed with water (300 mL×3) to afford crude Compound B2. The crude Compound B2 was dissolved in ethyl acetate (˜500 mL) and washed with brine (50 mL×3). The obtained organic phase was concentrated under reduced pressure to give a solid. To this solid was n-heptane/ethyl acetate (100 mL, v/v=100/1) and the mixture stirred at 25° C. for 2 hours. The resulting mixture was filtered and the filter cake was dried in vacuo at 40° C. for 12 hours. Compound B2 (107 g) was obtained as an off-white solid in 74.3% yield, 97.99% purity.

### Example 13. Synthesis of Compound B3

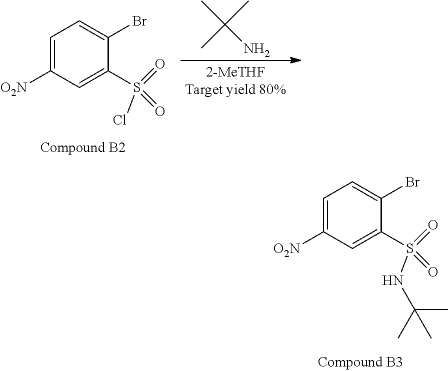

Compound B2 (75.2 g, 1.0 eq.) and 2-MeTHF (676 g) were charged to a suitable reactor, stirred and cooled to 0-10° C. under a nitrogen atmosphere. Tert-butylamine (41.2 g, 2.2 eq.) was charged over the course of 2.5 hours. After stirring for a further 6 hours at 20-30° C., >99% conversion to Compound B3 was seen.

The pH value of the mixture was adjusted to 1 (target: 1-2) with 1N HCl aqueous solution (362 g). After stirring the layers were separated and the 2-MeTHF layer was washed with water (320 g).

The 2-MeTHF was concentrated under vacuum to 2-3 vols and isopropyl alcohol (306 g, 4.1×) was added. Concentration to 2˜3 vols and addition of isopropyl alcohol were repeated for three times. The mixture was then heated to 60-70° C., agitated for 2 hours, cooled to 0-5° C. over the course of 6 hours and agitated at 0-5° C. for 4 hours. The mixture was filtered and the filter cake was rinsed with isopropyl alcohol (101 kg, 1.3 w/w) to give 72.40 kg of wet Compound B3. The product was dried at 40-50° C. for 24 hours to give 70.15 kg of Compound B3 with a purity of 99.7%, 99.3% assay and in 83% yield.

Alternate solvents that can be used for this reaction include but are not limited to ethyl acetate.

### Example 14. Synthesis of Compound B4

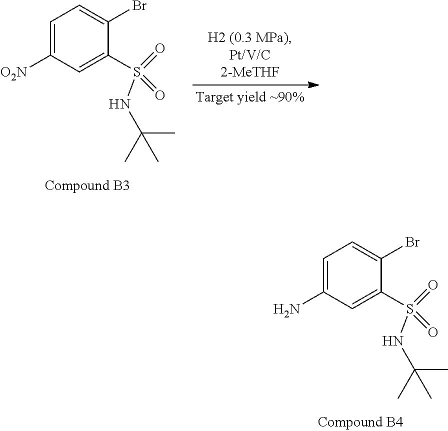 [SMILES: CC(C)(C)NS(=O)(=O)c1cc([N+](=O)[O-])ccc1Br ; CC(C)(C)NS(=O)(=O)c1cc(N)ccc1Br]

Compound B3 (69.9 g, 1.0 eq.), 2-MeTHF (674 g) and Pt/V/C catalyst (2% Pt, 1% V, 50% water wet) (2.6 g,) were charged to an appropriate hydrogenation reactor. Three vacuum-N2 degas cycles were performed before replacement with hydrogen. The pressure in reactor was kept at 0.28-0.38 MPa and the mixture was adjusted to 45-55° C. and stirred. After 14 hours, the mixture was cooled to 15-30° C. and a sample showed >99% conversion.

The mixture was filtered through diatomite filter to remove the catalyst and rinsed with 2-MeTHF (280 g). The combined filtrates were concentrated under vacuum to 2-3 vols keeping the internal temperature below 45° C. and then heated to 45-55° C. Heptane (420 g) was charged over the course of 4 hours for crystallization. The mixture was stirred at 45-55° C. for 8 hours, cooled to 15-30° C. over 8 hours and stirred for a further 4 hours. Solid was collected by filtration and the filter cake was rinsed with Heptane (102 g). The wet Compound B4 was dried at 40-50° C. for 24 hours to afford 60.75 g of Compound B4 with a purity of 99.7%, 100.0% assay and in 95% yield.

Alternate catalysts that can be used for this transformation include but are not limited to: 5% Pt/C, 5% Pt/C modified with H₃PO₂ and NH₄VO₃, and Fe/NH₄Cl (transfer hydrogenation).

### Example 15. Synthesis of Compound B5

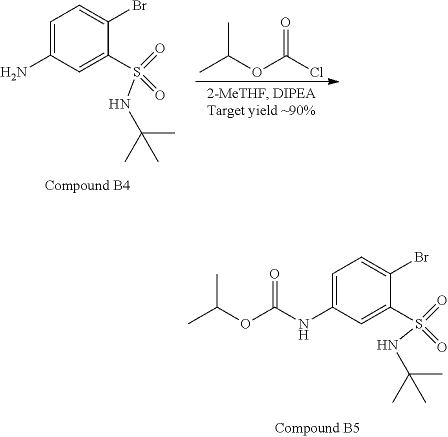

Compound B4 (60.7 g, 1.0 eq.) and 2-MeTHF (419 g) were charged to a suitable reactor and the mixture was adjusted to 15-25° C. and stirred under a nitrogen atmosphere. Then di-isopropyl ethylamine (DIPEA) (50.6 g, 2.1 eq.) was charged over 1 hour followed by isopropyl chloroformate (29.0 g, 1.2 eq.) over 2 hours. The mixture was heated to 65-75° C. and stirred for 6 h. After cooling to 25-40° C., a sample showed >99% conversion.

1N HCl aq solution (425 g) was used to quench the reaction mixture, to pH 1˜2. After layer separation, the 2-MeTHF solution was washed with water (180 mL) and then concentrated under volume to 2˜ 3 vols. 2-MeTHF (300 g) was added and the mixture was concentrated again to 2˜ 3 vols. The mixture was heated to 45-55° C., Heptane (518 g) was charged over 7 hours. The slurry was cooled to 15-25° C. and stirred a further 1-24 hours prior to filtration. The filter cake was washed with heptane (89 g). After drying at 35-45° C. for 24 hours 73.6 g of Compound B5 was isolated with a purity of 100%, 99.7% assay and in 94% yield.

### Example 16. Synthesis of Compound B6

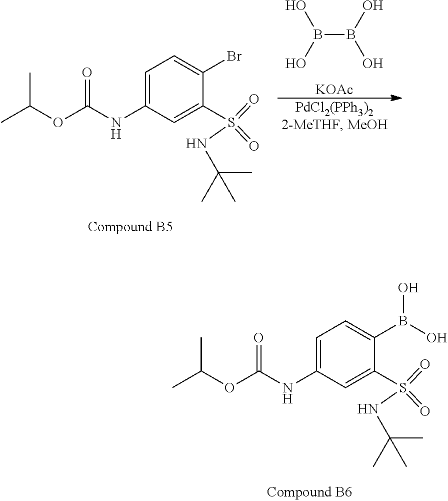 [SMILES: CC(C)OC(=O)Nc1ccc(Br)c(S(=O)(=O)NC(C)(C)C)c1 ; OB(O)B(O)O ; "PdCl2(PPh3)22-MeTHF, MeOH" ; CC(C)OC(=O)Nc1ccc(B(O)O)c(S(=O)(=O)NC(C)(C)C)c1 ; "KOAc"]

Compound B5 (72 g, 1 eq), potassium acetate (36.4 g, 2.1 equiv), 2-MeTHF (730 g) and MeOH (352 g) were charged to a suitable reactor which was evacuated and then nitrogen purged. The mixture was warmed to 55-65° C. with stirring under the nitrogen atmosphere and a methanolic solution of tetrahydroxydiboron (30 g in 356 g methanol, 1.8 eq.) was charged over 30 minutes followed by the addition of the slurry of Pd(Ph₃)₂Cl₂ in 2-MeTHF (0.64 g in 42 g 2-MeTHF, 0.005 eq.). The mixture was stirred at 55-65° C. for 15-24 hours.

Upon reaction completion, the mixture was cooled to 15-25° C. and filtered. The filtrate was recirculated through a CUNO™ filter for 6 h at 15-25° C. for decoloration, and CUNO™ was rinsed with EtOAc twice (2×134 g). The combined organic solution was concentrated under vacuum to 7-9 vols. EtOAc (532 g) was charged and the mixture was concentrated to 7˜ 9 vols followed by a second addition of ethyl acetate (532 g) and distillation to 7-9 vols. The EtOAc solution was washed with water (280 mL) and then twice with 25% NaCl aqueous solution (322 g and 140 g).

The EtOAc solution was concentrated to 3-4 vols under vacuum. Heptane (428 g) was charged at 15-25° C. over 5 hours and the slurry was stirred for 3 hours prior to cooling to 0-10° C. After further stirring at 0-10° C. for 8 hours, the mixture was filtered and the filter cake was rinsed with Heptane (100 g). The product was dried at 30-40° C. for 12 hours to give, 59.74 g Compound B6 in 99.1% purity, 96.1% assay and in 87% yield.

Other solvents that can be used for this reaction include but are not limited to methanol, ethanol, iso-propanol, dioxane, dimethylacetamide, 2-methylTHF, toluene, or a mixture thereof.

### Example 17. Alternatives to the Synthesis of Compound B6

Reaction of Compound B5 with B₂Pin₂ in the presence of palladium acetate and XPhos in DMF affords the boronate ester which is expected to react with Compound X in a comparable manner to Compound B6.

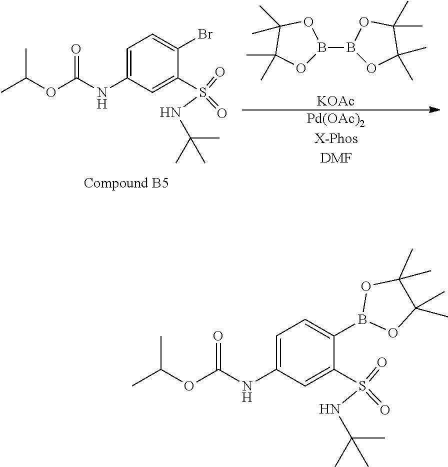

### Example 18. Synthesis of Compound A

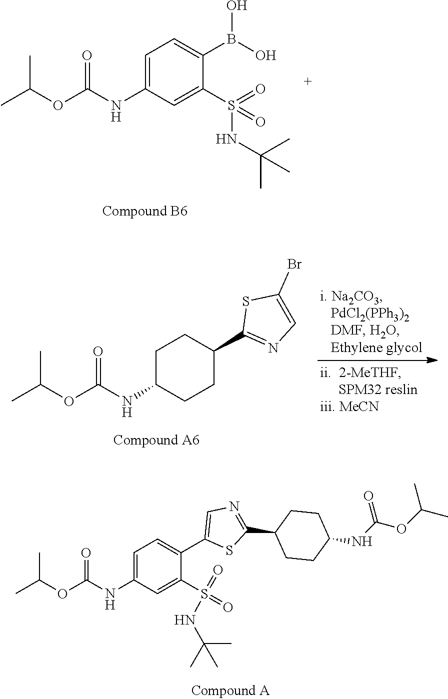

Compound A6 (43.4 g, 1.0 eq.), Compound B6 (48.9 g, 1.1 eq.), ethylene glycol (24 g, 3 eq.), DMF (615 g), and bis(triphenylphosphine)palladium (II) dichloride (4.3 g, 0.05 eq.) were charged to a suitable reactor which was evacuated and purged with nitrogen. An aqueous solution of sodium carbonate (39.9 g in 201 mL of water, 3.0 eq.) was charged into the reactor at 20-30° C. over 1 hour and the reaction mixture was heated to 70-80° C. and stirred at 70-80° C. under a nitrogen atmosphere for 2-3 h after which time>99% conversion was achieved.

The reaction mixture was cooled to 15-25° C., water (86 mL) was charged over 3 hours and then the mixture was stirred at this temperature for 20 hours. The solids were filtered and the filter cake was rinsed with water three times (200 mL each) and MTBE twice (80 mL each). The crude Compound A and SPM32 resin (20.9 g) were suspended in 2-MeTHF (857 g) and then heated to 60-70° C. and stirred at this temperature for 24 hours. The resin was removed by filtration, the filtrate was washed with water twice (217 mL) to provide a 2-MeTHF solution of Compound A.

The 2-MeTHF solution was concentrated to 4-6 vols. MeCN (538 g) was charged and the mixture was concentrated under vacuum to final volume of 9-11 vols. A second portion of MeCN (526 g) was charged and the mixture was concentrated again to 9-11 vols.

The slurry was heated to 70-80° C. and stirred at this temperature until full dissolution was achieved. The mixture was cooled to 45-55° C., optional Compound A seed (0.4 g) was added, followed by agitation at 45-55° C. for 3 hours. The mixture was further cooled to 15-25° C. over 2 hours, and agitated at this temperature for 7 hours then finally cooled to −5˜5° C. over 3 hours, and stirred at this temperature for 5 hours prior to filtration. The filter cake was rinsed with MeCN twice (102 g and 140 g). The solids were dried at 30-40° C. for 17 hours to give 56.95 g of Compound A with a purity of 100%, 99.5% assay and in 79.5% yield.

### Example 19. Alternate Approaches to Compound A

1. The boronic ester is expected to couple with Compound A6 to give Compound A in a comparable manner to the preferred process.

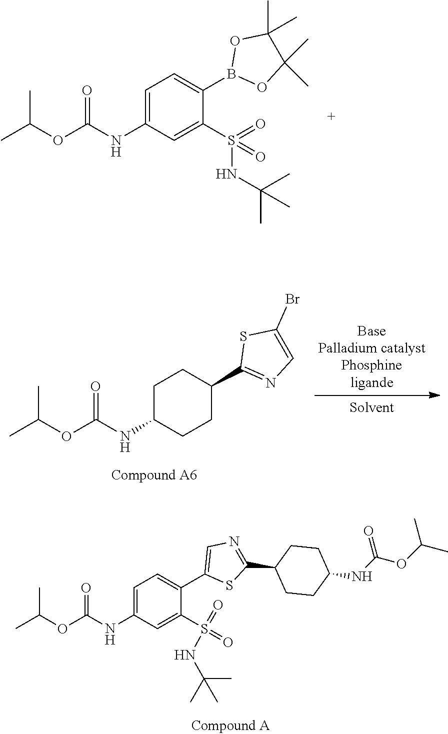

2. The coupling partners may be reversed

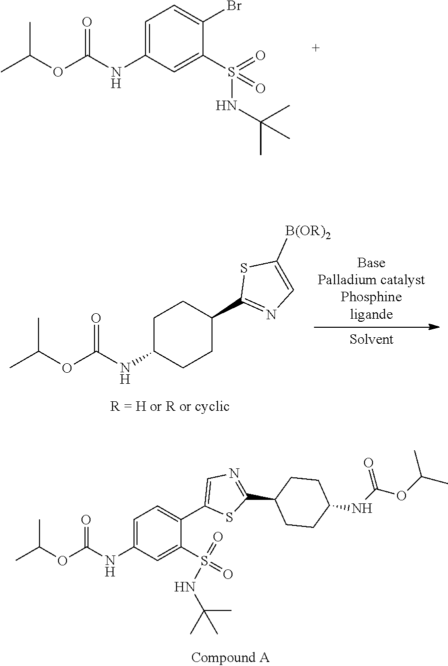

3. Compound A can be prepared from Compound Z. The synthesis of Compound Z is documented in U.S. Pat. No. 10,590,122

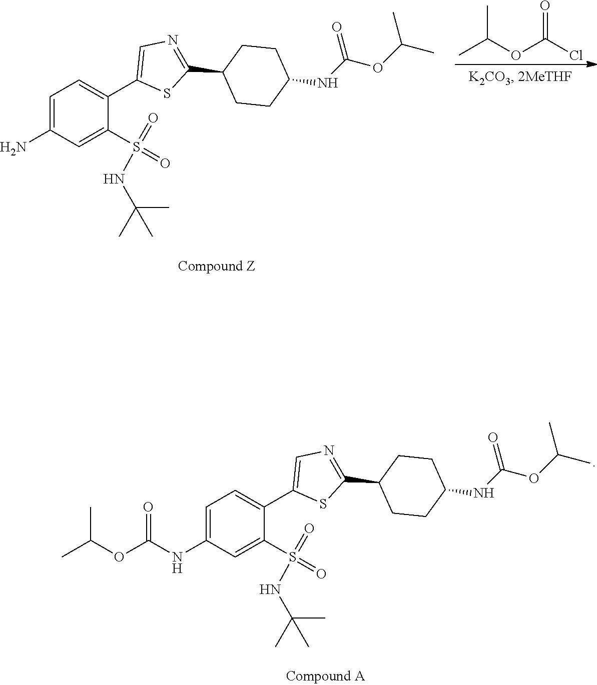

### EQUIVALENTS

The details of one or more embodiments of the invention are set forth in the accompanying description above. Although any methods and materials similar or equivalent to those described herein can be used in the practice or testing of the present application, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the invention to the precise form disclosed, but by the claims appended hereto.

1. A method for preparing Compound A: 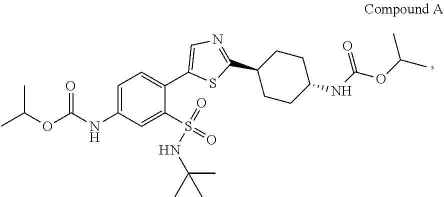 [SMILES: CC(C)OC(=O)Nc1ccc(-c2cnc([C@H]3CC[C@H](NC(=O)OC(C)C)CC3)s2)c(S(=O)(=O)NC(C)(C)C)c1 ; ","]
or a pharmaceutically acceptable salt or solvate thereof, comprising Step (a) or Step (b):
Step (a): reacting Compound X with Compound Y to form Compound A: 5 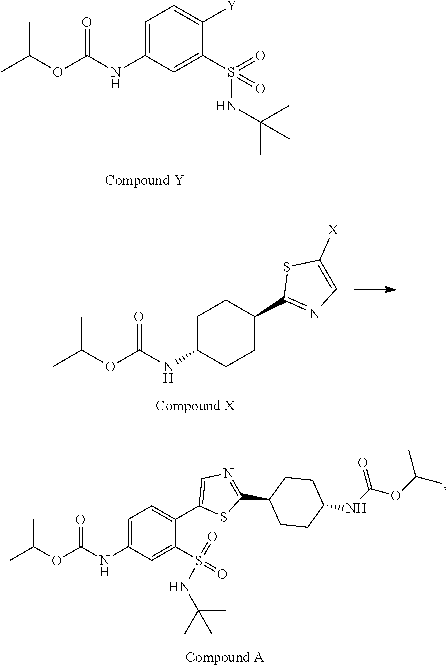
or
Step (b): reacting Compound Xₐₜ with Compound Yₐₜ to form Compound A: 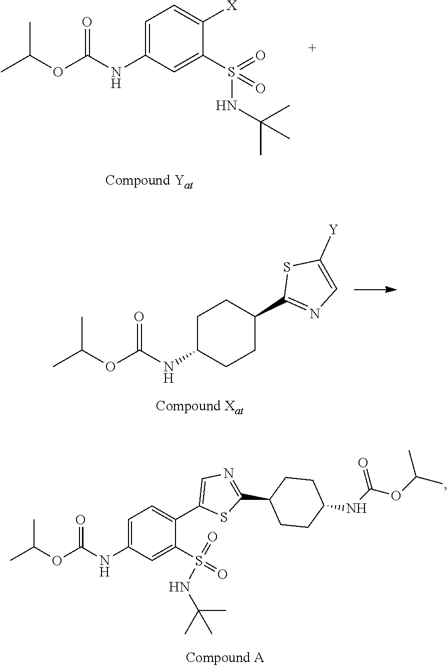
wherein X is —Cl, —Br, or —I and Y is

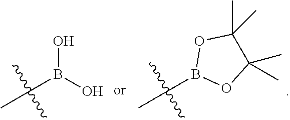

2. The method of claim 1, comprising Step (a).

3. The method of claim 1 or 2, wherein Step (a) is Step (a1):
Step (a1): reacting Compound X with Compound B6 to form Compound A:

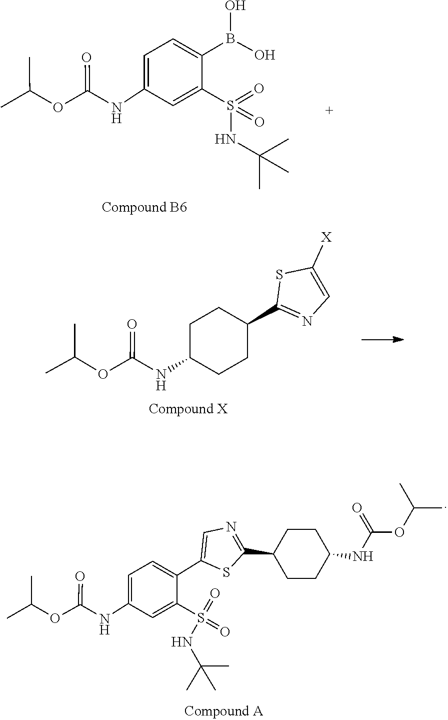

4. The method of claim 1 or 2, wherein Step (a) is Step (a2):
Step (a2): reacting Compound X with Compound B6′ to form Compound A:

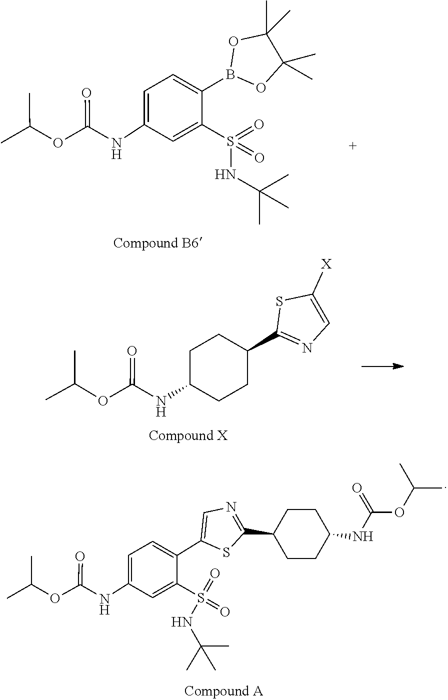

5. The method of any one of claims 1-4, wherein X is —Br.

6. The method of any one of claims 1-5, further comprising Step A4:
Step A4: cyclization of Compound A4: 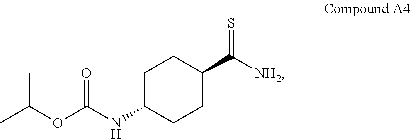 [SMILES: CC(C)OC(=O)N[C@H]1CC[C@H](C(N)=S)CC1]
to form Compound A5:

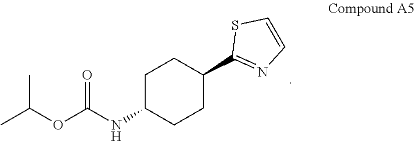 [SMILES: CC(C)OC(=O)N[C@H]1CC[C@H](c2nccs2)CC1]

7. The method of claim 6, comprising cyclization of Compound A4 in the presence of an acid or a Lewis acid selected from ZnBr₂, Yb(OTf)₃·H₂O, Y(OTf)₃, Sc(OTf)₃, Zn(OTf)₂, FeCl₂, CoCl₂, p-toluene sulfonic acid, and camphor sulfonic acid, or a combination thereof.

8. The method of claim 6 or 7, comprising cyclization of Compound A4 in the presence of 2-bromo-1,1-dimethoxy-ethane; 2-bromo-1,1-diethoxyethane; 2-bromoacetaldehyde; or 2-chloroacetaldehyde; or a combination thereof.

9. The method of any one of claims 6-8, wherein Step A4 is as follows:

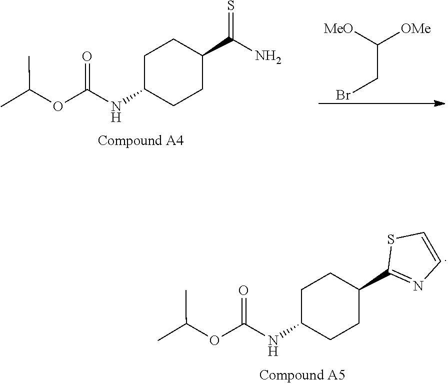 [SMILES: CC(C)OC(=O)N[C@H]1CC[C@H](C(N)=S)CC1 ; COC(CBr)OC ; CC(C)OC(=O)N[C@H]1CC[C@H](c2nccs2)CC1]

10. The method of any one of claims 6-9, comprising cyclization of Compound A4 in the presence of ZnBr₂, Yb(OTf)₃·H₂O, Y(OTf)₃, or Sc(OTf)₃, or a combination thereof.

11. The method of any one of claims 6-10, comprising cyclization of Compound A4 in the presence of ZnBr₂.

12. The method of any one of claims 6-11, wherein Step A4 forms Compound A5 and Compound SP₁: 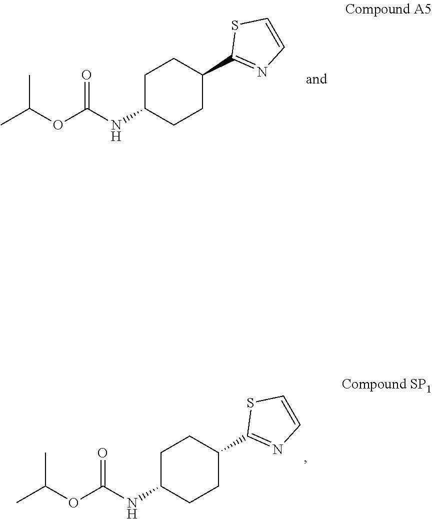 [SMILES: CC(C)OC(=O)N[C@H]1CC[C@H](c2nccs2)CC1 ; CC(C)OC(=O)N[C@H]1CC[C@@H](c2nccs2)CC1]
wherein the wt/wt ratio in the amount between Compound A5 and Compound SP1 is at least about 50:50, at least about 55:45, at least about 60:40, at least about 65:35, at least about 70:30, at least about 75:25, at least about 80:20, at least about 85:15, at least about 90:10, at least about 92:8, or at least about 95:5.

13. The method of any one of claims 6-12, wherein Step A4 forms Compound A5 and Compound SP₁: 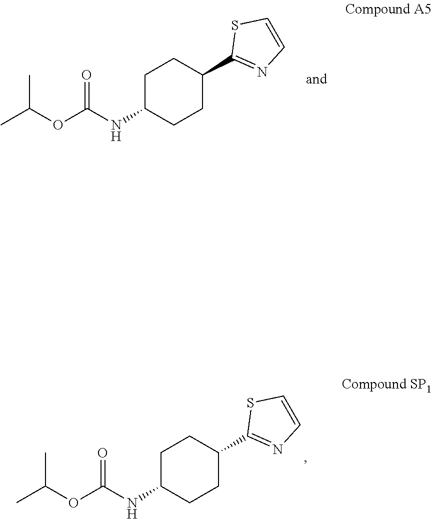 [SMILES: CC(C)OC(=O)N[C@H]1CC[C@H](c2nccs2)CC1 ; CC(C)OC(=O)N[C@H]1CC[C@@H](c2nccs2)CC1]
wherein the total yield of Compound A5 and Compound SP1 is at least about 40%, at least about 50%, at least about 55%, at least about 60%, at least about 65%, at least about 70%, at least about 75%, at least about 80%, at least about 85%, or at least about 90%.

14. The method of any one of claims 6-13, further comprising one or more steps of Step A1, Step A2, and Step A3:
Step A1: reacting Compound A1: 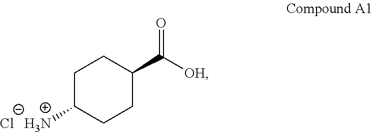
with isopropyl chloroformate to form Compound A2: 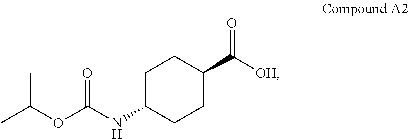 [SMILES: CC(C)OC(=O)N[C@H]1CC[C@H](C(=O)O)CC1]
Step A2: reductive amination of Compound A2 to form Compound A3: 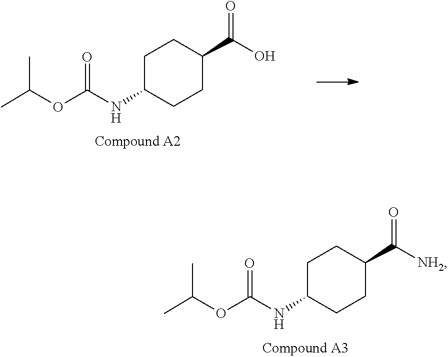 [SMILES: CC(C)OC(=O)N[C@H]1CC[C@H](C(=O)O)CC1 ; CC(C)OC(=O)N[C@H]1CC[C@H](C(N)=O)CC1]
and
Step A3: converting Compound A3 to Compound A4:

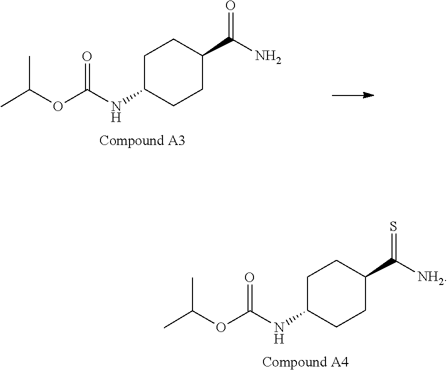 [SMILES: CC(C)OC(=O)N[C@H]1CC[C@H](C(N)=O)CC1 ; CC(C)OC(=O)N[C@H]1CC[C@H](C(N)=S)CC1]

15. The method of any one of claims 1-5, further comprising Step A4′:
Step A4′: hydrogenation of Compound Aₐₜ: 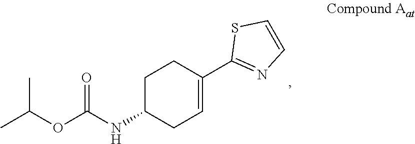 [SMILES: CC(C)OC(=O)N[C@H]1CC=C(c2nccs2)CC1]
to form Compound A5:

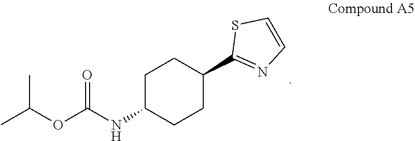 [SMILES: CC(C)OC(=O)N[C@H]1CC[C@H](c2nccs2)CC1]

16. The method of any one of claims 1-15, further comprising Step A5:
Step A5: reacting Compound A5: 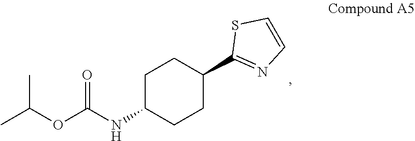 [SMILES: CC(C)OC(=O)N[C@H]1CC[C@H](c2nccs2)CC1]
with a halogenating agent to form Compound X:

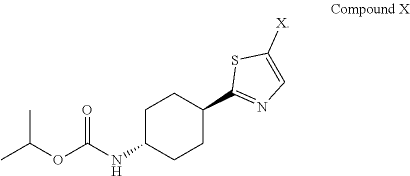

17. The method of any one of claims 1-5, further comprising Step A1, Step A2, Step A3, Step A4, and Step A5:
Step A1: reacting Compound A1: 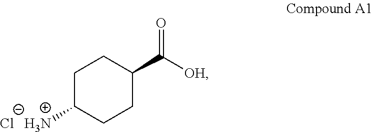
with isopropyl chloroformate to form Compound A2: 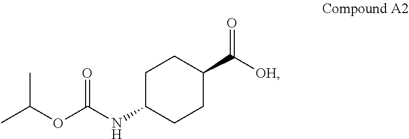 [SMILES: CC(C)OC(=O)N[C@H]1CC[C@H](C(=O)O)CC1]
Step A2: reductive amination of Compound A2 to form Compound A3: 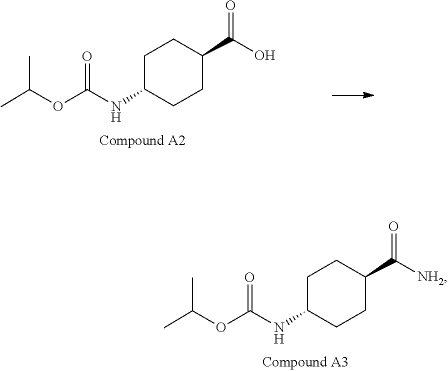 [SMILES: CC(C)OC(=O)N[C@H]1CC[C@H](C(=O)O)CC1 ; CC(C)OC(=O)N[C@H]1CC[C@H](C(N)=O)CC1]
Step A3: converting Compound A3 to Compound A4: 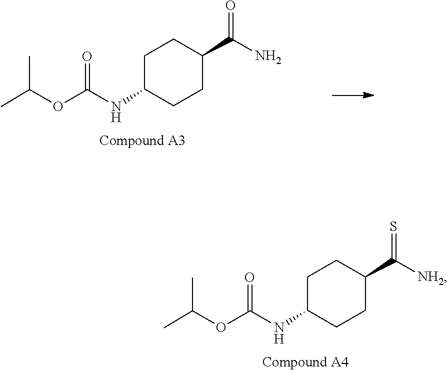 [SMILES: CC(C)OC(=O)N[C@H]1CC[C@H](C(N)=O)CC1 ; CC(C)OC(=O)N[C@H]1CC[C@H](C(N)=S)CC1]
Step A4: cyclization of Compound A4: 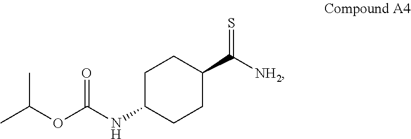 [SMILES: CC(C)OC(=O)N[C@H]1CC[C@H](C(N)=S)CC1]
to form Compound A5: 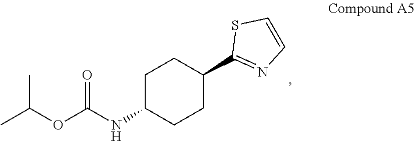 [SMILES: CC(C)OC(=O)N[C@H]1CC[C@H](c2nccs2)CC1]
and
Step A5: reacting Compound A5 with a halogenating agent to form Compound X:

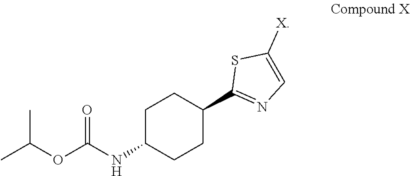

18. The method of claim 16 or 17, wherein the halogenating agent is a brominating agent.

19. The method of any one of claims 1-18, further comprising one or more steps of Step B1, Step B2, Step B3, Step B4, and Step B5:
Step B1: converting Compound B1 to Compound B2: 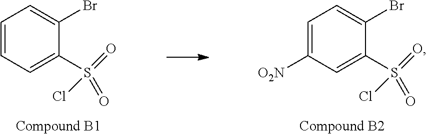 [SMILES: O=S(=O)(Cl)c1ccccc1Br ; O=[N+]([O-])c1ccc(Br)c(S(=O)(=O)Cl)c1]
Step B2: converting of Compound B2 to form Compound B3: 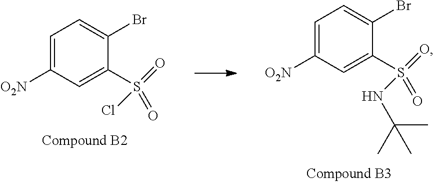 [SMILES: O=[N+]([O-])c1ccc(Br)c(S(=O)(=O)Cl)c1 ; CC(C)(C)NS(=O)(=O)c1cc([N+](=O)[O-])ccc1Br]
Step B3: reducing Compound B3 to Compound B4: 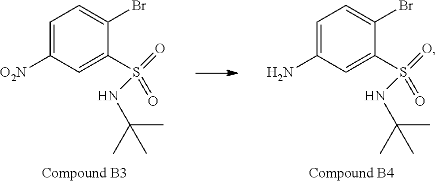 [SMILES: CC(C)(C)NS(=O)(=O)c1cc([N+](=O)[O-])ccc1Br ; CC(C)(C)NS(=O)(=O)c1cc(N)ccc1Br]
Step B4: converting of Compound B4 to form Compound B5: 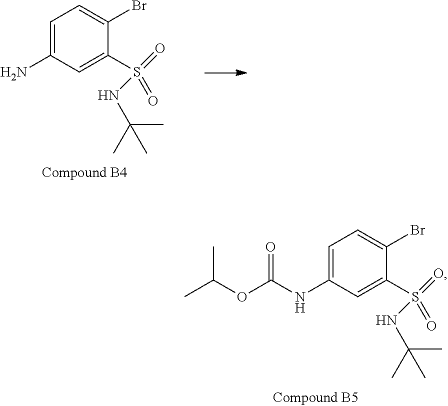 [SMILES: CC(C)(C)NS(=O)(=O)c1cc(N)ccc1Br ; CC(C)OC(=O)Nc1ccc(Br)c(S(=O)(=O)NC(C)(C)C)c1]
and
Step B5: converting of Compound B5 to form Compound Y:

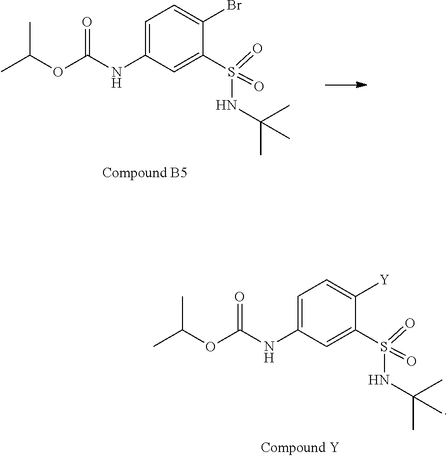

20. The method of claim 19, wherein Step B5 is as follows:

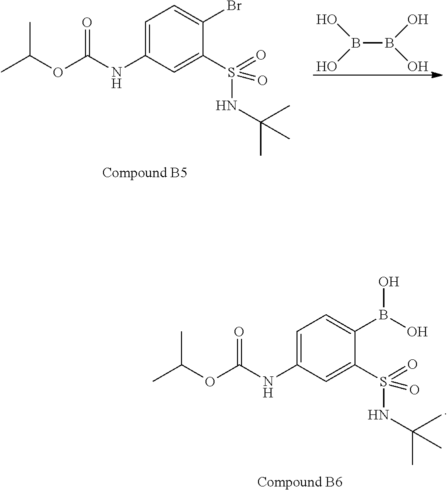

21. The method of claim 19, wherein Step B5 is as follows:

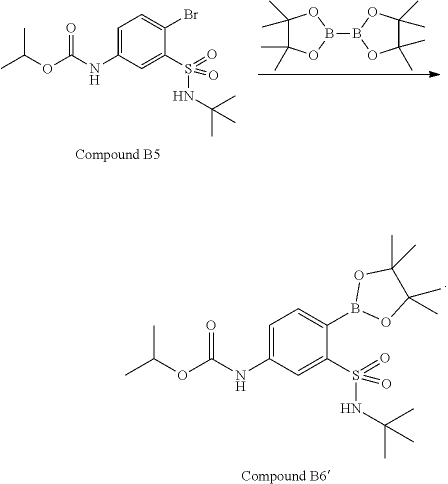

22. The method of any one of claims 1-21, wherein Step (a) or Step (b) is carried out in the presence of a palladium catalyst.

23. The method of claim 22, wherein the palladium catalyst is selected from: 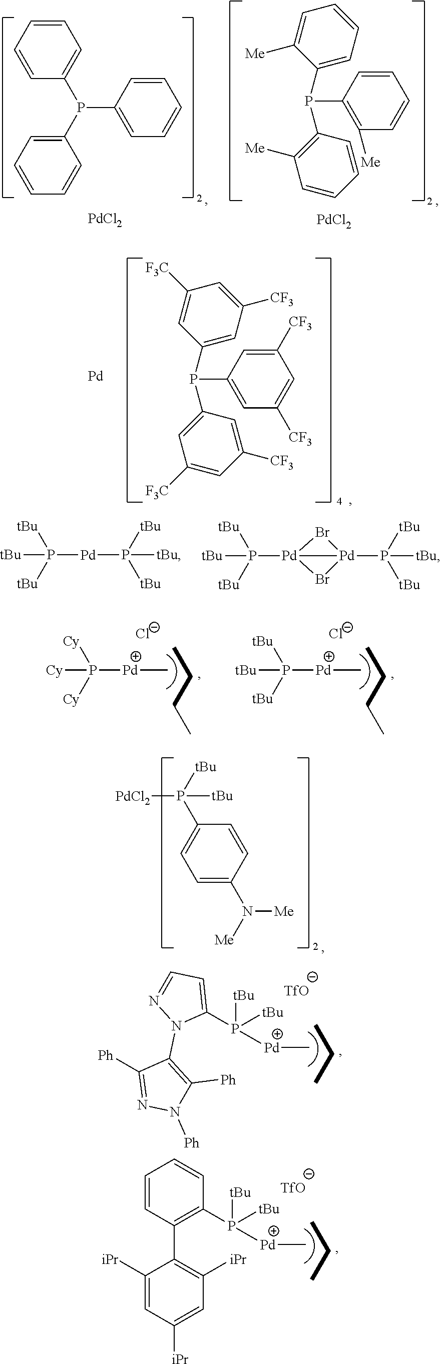 [chemical structure]
